(19) United States
(12) Patent Application Publication
(10) Pub. No.: US 2024/0010535 A1
(43) Pub. Date: Jan. 11, 2024

(54) COMPOSITIONS AND METHODS FOR RAPID DEGRADATION AND AMELIORATION OF MARINE OIL SPILLS

(71) Applicant: 683107 Alberta Ltd., Lethbridge (CA)

(72) Inventors: Jacob HØJGAARD, Lethbridge (CA); Thomas HOUGAARD, Lethbridge (CA); Malcolm LOWINGS, Lethbridge (CA); Michael LOWINGS, Lethbridge (CA); Camilla NESBØ, Lethbridge (CA); Peter STOUGAARD, Lethbridge (CA)

(21) Appl. No.: 18/255,240

(22) PCT Filed: Nov. 30, 2021
(86) PCT No.: PCT/CA2021/051711

§ 371 Date: May 31, 2023

Related U.S. Application Data
(60) Provisional application No. 63/119,788, filed on Dec. 1, 2020.

(51) Int. Cl. C02F 3/34

(52) U.S. Cl. CPC C02F 3/344; C02F 3/348; C02F 2101/32

(57) ABSTRACT

Disclosed herein are dynamic microbiome-based compositions for rapid degradation and amelioration of marine oil spills, methods for preparing the dynamic microbiome-based compositions, and methods for deploying the dynamic microbiome-based compositions into oil spills.

### TECHNICAL FIELD

The present disclosure generally relates to methods for degradation and amelioration of marine oil spills. In particular, the present disclosure relates to compositions for rapid deployment into marine oil spills for degradation of the hydrocarbons therein, methods and systems for preparing and producing the compositions, and methods and systems for deployment of the compositions into marine oil spills.

### BACKGROUND

There are numerous and ongoing incidents of small to medium size oil spills occurring in coastal marine waters as consequences of commercial marine transport, commercial fishing, and recreational boating. Common sources of marine oil spills include slow leakage from poorly maintained engines during operation, emptying of hydrocarbon-contaminated bilge waters and sludges, intentional dumping of unwanted fuel, leakage from abandoned derelict vessels, loading and unloading of cargo at port facilities, spillage at fuel docks during refueling, and from leakage and spill incidents at marine terminal facilities for hydrocarbon transmission pipelines. The ITOPF (International Tanker Owners Pollution Federation) classified small operational spill events as those where less than 7 metric tonnes were discharged, medium-scale spill events as those where between 7 to 700 metric tonnes were discharged, and large spill events resulting from a collision, hull failure, or explosion as those where over 700 metric tonnes were discharged.

In addition to the problems arising from influxes of hydrocarbon fuels from marine oil spill events, accumulations of spilled hydrocarbon fuels in coastal marine waters, harbours, terminals, and marinas occurring over extended periods of time, have extreme devastating long-term effects on marine species and ecologies, and typically, require decades to restore the marine environments to ecological health. Spill-response strategies typically focus on rapid containment of a spill accompanied by attempts to prevent its spreading over large surface areas of ocean waters and from washing ashore. Spill-response strategies also include separation and removal of spilled oil from water. However, these efforts are often overwhelmed by the sheer magnitude of the spills.

Consequently, hydrocarbon loading of nearshore and marine harbour waters typically accumulates and increases over extended periods of time resulting in long-term significantly negative effects on marine life and ecology. It is apparent that current strategies for responding to and ameliorating small and medium-size oil spills, and for remediating hydrocarbon-polluted harbours and terminals, are simply inadequate.

### SUMMARY

The embodiments of the present disclosure generally relate to biological response compositions, systems, and methods configured for deployment into marine oil spills for rapid degradation and dispersion of the spills. The biological response compositions disclosed herein may be prepared for and are suitable for deployment into and for rapid dispersion of marine oil spills that comprise primarily light crude oil or alternatively, heavy crude oil, or alternatively, heavy fuel oils, and mixtures thereof. The biological response compositions, systems, and methods are particularly suitable for deployment into small and medium-size spills.

According to some example embodiments, the biological response compositions disclosed herein may comprise combinations of selected dynamic microbiome-based components and carriers selected therefor. The biological response compositions may be capable of self-propulsion into and dispersion within an oil spill. According to some aspects, the selected microbiome components may be enriched by methods disclosed herein, prior to combining with selected carriers therefor. The microbiome components disclosure herein comprise complex naturally occurring mixtures of biological species collected from selected marine waters and selected for their ability to consume straight-chain and/or branched and/or aromatic hydrocarbons as energy and/or nutrient sources. According to some aspects, the selected microbiome components may be enriched and stored at or below −70° C. or maintained for extended periods of time by continued culturing in fluid media supplemented with supplies of selected straight-chain and/or branched and/or aromatic hydrocarbons.

Also disclosed herein are example methods for the preparation and maintenance of the selected microbiome components. Large volumes of the biological response compositions disclosed herein may be rapidly prepared after the occurrence of a marine spill, for deployment thereinto as quickly as possible.

Some embodiments of the present disclosure relate to systems and methods for deploying the biological response compositions disclosed herein, into an oil spill. The systems may include, among other things, containers for transporting the biological response compositions to ocean oil spill locations wherein the biological activities of enriched hydrocarbon-degrading microbiomes in the compositions increase and proliferate during transport. The systems may include equipment and mechanisms for targeted discharge of the biological response compositions into and within a marine oil spill. According to some aspects, the targeted discharges may be pressurized or alternatively, unpressurized. According to some aspects, the target discharges may use the same types of pressurizing equipment and nozzles that are used for deployment of chemical dispersants.

Other embodiments of the present disclosure relate to biological remediation compositions, methods, and compositions configured for deployment into long-term heavily polluted nearshore marine environments such as harbours, ports, and the like, whereon marine vessels are moored for periods of time prior to or after docking for discharge or loading of passengers or cargo. Such long-term heavily polluted nearshore marine environments are known to comprise very high levels of hydrocarbons in their water columns and sediments.

According to some example embodiments, the biological response compositions disclosed herein may comprise combinations of dynamic microbiome-based components that may be isolated from water columns and/or sediment of highly polluted nearshore marine sites, then enriched and maintained in fluid media supplemented with straight-chain and/or branched and/or aromatic hydrocarbons. The enriched microbiome-based components may be combined with selected carriers for deployment on a regular basis, into a water column of a long-term highly polluted nearshore marine site.

### BRIEF DESCRIPTION OF THE DRAWINGS

These and other features of the present disclosure will become more apparent in the following detailed description in which reference is made to the appended drawings. The appended drawings illustrate one or more embodiments of the present disclosure by way of example only and are not to be construed as limiting the scope of the present disclosure.

FIG. 1 is a chart showing microbial growth measured as changes in optical density (OD) at 600 nm, in fuel oil-amended seawater receiving enriched oil-degrading microbiomes combined with a copepod carcasses carrier;

FIG. 2 is a chart showing microbial growth, measured as changes in OD, in fuel oil-amended seawater receiving enriched oil-degrading microbiomes combined with a copepod eggs carrier;

FIG. 3 is chart showing microbial growth, measured as changes in OD, in fuel oil-amended seawater receiving enriched oil-degrading microbiomes combined with a microalgae carrier;

FIG. 4 shows charts illustrating the effects of an “active biological countermeasures” (ABCM) treatment and an enriched microbiome on the degradation of o-xylene (FIG. 4A), a mixture of m-xylene and p-xylene (FIG. 4B), and ethylbenzene (FIG. 4C) in seawater samples, while FIG. 4D is a table showing the reductions in these hydrocarbons compared to natural degradation processes;

FIG. 5 shows charts illustrating the effects of an ABCM treatment and an enriched microbiome on the degradation of naphthalene (FIG. 5A), benzene (FIG. 5B), and toluene (FIG. 5C) in seawater samples, while FIG. 5D is a table showing the reductions in these hydrocarbons compared to natural degradation processes;

FIG. 6 is a schematic illustration of a test barrel configuration used in Example 3;

FIG. 7 are charts showing changes in pH (FIG. 7A) and oxidative reduction potential (FIG. 7B) measurements over a 26-day time period in the barrel study disclosed in Example 3;

FIG. 8 are charts showing changes in turbidity (FIG. 8A) and optical density (FIG. 8B) measurements over a 26-day time period in the barrel study disclosed in Example 3;

FIG. 9A is a chart showing patterns of hydrocarbon degradation in seawater receiving an oil slick and an ABCM-inoculated kelp treatment in Example 3;

FIG. 9B is a chart showing patterns of hydrocarbon degradation in seawater receiving an oil slick and an ABCM-inoculated microalgae treatment in Example 3;

FIG. 9C is a chart showing patterns of hydrocarbon degradation in seawater receiving an oil slick and an ABCM treatment in Example 3;

FIG. 9D is a chart showing patterns of hydrocarbon degradation in a control seawater barrel receiving an oil slick only in Example 3;

FIG. 9E is a chart showing patterns of hydrocarbon degradation in seawater receiving an oil slick and an ABCM-inoculated copepod carcass treatment in Example 3;

FIG. 9F is a chart showing patterns of hydrocarbon degradation in seawater receiving an oil slick and a copepod carcass treatment in Example 3;

FIG. 10 is a GC-FID chart showing the presence of a mixture of alkanes in water sampled 1 day after crude oil was added to the test barrels in Example 3;

FIG. 11 is a GC-FID chart from a water sample collected 18 days after crude oil was added to the test barrel receiving an ABCM composition with copepod carcasses as the carrier in Example 3;

FIG. 12A is a chart illustrating microbial population community diversity determined with 16S rRNA gene amplicon analyses at the beginning of the 4-week study in Example 3 at Day 0, Day 8, and Day 15;

FIG. 12B is a chart illustrating the changes in microbial population community succession and diversity as determined with 16S rRNA gene amplicon analyses at Day 22 and at the end of the 4-week study in Example 3;

FIG. 12C is a colour coding chart identifying microbial populations shown in FIGS. 10A and 10B as determined with 16S rRNA gene amplicon analyses of samples collected during the 4-week study in Example 3;

FIG. 13A is a chart prepared from the data shown in FIGS. 12A-12C, illustrating the effects of ABCM treatments on microbial population community diversity determined with 16S rRNA gene amplicon analyses over a 29-day strudy period in Example 3;

FIG. 13B is a chart prepared from the data shown in FIGS. 12A-12C, illustrating the effects of ABCM treatments on microbial population community diversity determined with 16S rRNA gene amplicon analyses over a 29-day strudy period in Example 3;

FIG. 14 is a chart showing a non-metric dimensional scaling of the 16S rRNA gene data collected from the six treatments at the end of the barrel study disclosed in Example 3;

FIG. 15 is a chart showing changes in pH measurements over the first days in the barrel study disclosed in Example 4;

FIG. 16 is a chart showing changes in optical density measurements over the first 15 days in the barrel study disclosed in Example 4;

FIG. 17 are charts showing the biodegradation of (i) an oil slick in a control barrel (FIG. 17A), an oil slick in a barrel that received an ABCM treatment (FIG. 17B), and (iii) an oil slick in a barrel that received an ABCM treatment in a carrier (FIG. 17C), in Example 4;

FIG. 18 are charts showing the breakdown products present 21 days after an oil slick was added to (i) a control barrel (FIG. 18A), (ii) a barrel that received an ABCM treatment (FIG. 18B), and (iii) a barrel that received an ABCM treatment in a carrier (FIG. 18C), in Example 4;

FIG. 19 are GC-FID charts showing the presence of alkanes in water samples collected from (i) a control barrel (plotted in blue), (ii) a barrel that received an ABCM treatment (plotted in red), and (iii) a barrel that received an ABCM treatment in a carrier (plotted in green), 8 days after (FIG. 19A) and 24 15 days after (FIG. 19B) addition of an oil slick to each barrel in Example 4;

FIG. 20 is a chart showing a non-metric dimensional scaling of the 16S rRNA gene data collected from the five treatments at the end of the barrel study disclosed in Example 4;

FIG. 21A is a chart illustrating microbial population community diversity development determined with 16S rRNA gene amplicon analyses over a 38-day period in Example 4, in barrels containing (i) seawater only (barrel A1, negative control) and (ii) seawater overlaid with an oil slick (barrel B2, positive control);

FIG. 21B is a chart illustrating microbial population community diversity development in barrels containing (iii) seawater that received an ABCM treatment in a carrier prior to application of an oil slick (barrels E1, E2, E3);

FIGS. 21C, 21D, 21E are colour coding charts identifying microbial populations shown in FIGS. 21A and 21B as determined with 16S rRNA gene amplicon analyses of samples collected during the 38-day study in Example 4;

FIG. 22A are charts showing the relative abundance (%) of Sulfitobacter, Flavobacteriaceae, and Psychroserpens populations in treatment barrels A1, B2, C1, D1, E1, E2, and E3 in Example 4;

FIG. 22B are charts showing the relative abundance (%) of Alcanivorax, Colwellia, and Bacteroidetes populations in treatment barrels A1, B2, C1, D1, E1, E2, and E3 in Example 4;

FIG. 22C are charts showing the relative abundance (%) of Oleispira, Cycloclasticus, and Amphritea populations in treatment barrels A1, B2, C1, D1, E1, E2, and E3 in Example 4;

FIG. 23A is a chart illustrating microbial population community diversity development in seawater samples collected dockside at the Westridge Marine Terminal in Burrard Inlet in Burnaby, BC, determined with 16S rRNA gene amplicon analyses after the seawater samples were collected for Example 5;

FIG. 23B is a colour coding chart identifying microbial populations shown in FIG. 21A as determined with 16S rRNA gene amplicon analyses of seawater samples collected for Example 5;

FIG. 24 is a chart showing changes in optical density measurements over a 60-day period in the study disclosed in Example 5;

FIG. 25A is a chart illustrating microbial population community diversity development determined with 16S rRNA gene amplicon analyses over a 38-day period in ABCM cultures enriched in Alberta conventional crude oil in Example 5;

FIG. 25B is a chart illustrating microbial population community diversity development determined with 16S rRNA gene amplicon analyses over (i) a 20-day period in ABCM cultures enriched in diesel oil, (ii) a 43-day period in ABCM cultures enriched in bunker C oil, and (iii) a 56-day period in ABCM cultures enriched in diluted bitumen, in Example 5;

FIG. 25C is a colour coding chart identifying microbial populations shown in FIGS. 23A and 23B as determined with 16S rRNA gene amplicon analyses of ABCM microbiomes enriched on selected forms of crude oils in Example 5;

FIG. 26 is a chart showing a non-metric dimensional scaling of the 16S rRNA gene data collected from the four ABCM treatments disclosed in Example 5;

FIG. 27 is a chart showing the % abundances of selected genes involved in degradation of aliphatic hydrocarbons in Tromsø (North Atlantic) Samples 1 to 7;

FIG. 28 is a chart showing the % abundances of selected genes involved in degradation of aromatic hydrocarbons in Tromsø (North Atlantic) Samples 1 to 7;

FIG. 29 is a chart showing the % abundances of selected genes involved in degradation aliphatic and aromatic hydrocarbons in BC (North Pacific) Samples 8-10;

FIG. 30 is a chart showing a sequence network of Alcanovorax species in Tromsø (North Atlantic) Samples 1 to 7 and BC (North Pacific) Samples 8-10;

FIG. 31 is a phylogenic tree of Porticoccaceae genomes in Tromsø (North Atlantic) Samples 1 to 7 and BC (North Pacific) Samples 8-10; and

FIG. 32 is a phylogenic tree of Cycloclasticus genomes in Tromsø (North Atlantic) Samples 1 to 7 and BC (North Pacific) Samples 8-10.

### DETAILED DESCRIPTION

In the present disclosure, all terms referred to in singular form are meant to encompass plural forms of the same. Likewise, all terms referred to in plural form are meant to encompass singular forms of the same. Unless defined otherwise, all technical and scientific terms used herein have the same meaning as commonly understood by one of ordinary skill in the art to which this disclosure pertains.

As used herein, the term “microbiome” refers to all of the microbial species present in a marine water column environment, and includes the microbial species present in marine snow and in the upper sediment layers of ocean floors. Additionally, the term “microbiome” encompasses the activity of the microbial species that results in the formation of specific ecological niches. A microbiome will form a dynamic and interactive micro-ecosystem that is prone to change in time and scale, and which, may be integrated in into selected macro-ecosystems.

As used herein, the term “microbial species” refers to all viruses, bacteria, archaea, fungi, and yeasts that are present in one or more samples collected from a marine water column. “Microbial species” may also be referred to herein as “microbial populations” or also as “microbial communities”.

As used herein, the term “enrichment” refers to the culturing of a microbiome obtained from a sample of marine water in a selected carbon-fee medium or alternatively, a selected carbon-limited medium supplemented with a selected hydrocarbon, to select for and increase the abundance and biological activity of microbial species with the capacity to tolerate and degrade the selected hydrocarbon.

As used herein, the term “hydrocarbon-enriched microbiomes” means that the microbiomes produced with the enrichment methods disclosed herein will comprise microbial communities composed of microbial species that are capable of flourishing on hydrocarbons as the only or the primary sources of carbon nutrients.

As used herein, the term “dynamic microbiome-based composition” refers to a composition wherein a hydrocarbon-enriched microbiome has been combined with a selected carrier in a selected liquid medium, and is suitable for deployment into a marine oil spill. The term “dynamic” means that the species composition of the hydrocarbon-enriched microbiome is not static or constant but rather, will continuously evolve whereby the numbers of each individual hydrocarbon-degrading microbial specie present in the microbiome at the time of preparation of the composition, may subsequently increase and/or decrease over a period of time prior to deployment of the dynamic microbiome-based composition into a marine oil spill.

As used herein, the term “marine water column” refers to a conceptual column of water from the surface of a selected location of an ocean to its bottom sediment. Those skilled in this art will understand that marine water columns are commonly divided into five parts (also referred to as pelagic zones) wherein (i) the first zone is from the ocean surface to 200 meters below the surface and is referred to as the “epipelagic zone”, (ii) the second zone is from 200 to 1000 meters below the surface and is referred to as the “mesopelagic zone”, (iii) the third zone is from 1000 to 4000 meters below the surface and is referred to as the “bathypelagic zone”, (iv) the fourth zone is from 4000 meters below the surface to the level seafloor and is referred to as the “abyssopelagic zone”, and (v) the fifth zone pertains to depressions and crevices below the level seafloor and is referred to as the “hadopelagic zone”. Those skilled in this art will also understand that each pelagic zone in a marine water column comprises different types of microbial species associated with the depth of the zone, and the geographical location of the marine water column (e.g., tropical, temperate, cold water regions associated with proximity to the North and South Poles). Marine water column samples collected from near-offshore sites will typically comprise microbial species resident in epipelagic zones that are free of chronic hydrocarbon pollution, whereas water column samples collected from marine harbors will comprise microbial species from epipelagic zones that have acclimated to utilize hydrocarbons as primary nutrient and energy sources.

As used herein, the term “marine snow” refers to a continuous shower of organic detritus that falls from the upper layers through the lower layers of a marine column and eventually settles on the ocean floor.

As used herein, the term “marine oil snow” refers to the commingling and adsorption of marine snow to oil droplets and globules that infiltrate a water column. The organic detritus may chemically interact with the oil droplets and globules to form a more diverse array of oxygenated hydrocarbon molecules that settle onto ocean floor sediments. The oxygenated hydrocarbon molecules in marine snow generally are more toxic to organisms and microorganisms in sediments than are non-oxygenated hydrocarbon molecules.

As used herein, the term “ocean floor sediment” refers to layers of solids at the bottom of marine water columns, generally comprising mixtures of particles that include (i) terrigenous mineral and inorganic particles originating from land adjacent to oceans, (ii) biogenic particles comprising organic and inorganic material released from decomposing microorganisms, aquatic species, and fecal pellets from zooplankton, and (iii) authigenic particles that are formed in place on the ocean floor by way of chemical reactions and microbiological activity. Ocean floor sediments are rather deep and range from 150 m to more than 10 km.

As used herein, the term “metagenome” refers to the genomic materials of a microbiome from environmental samples, enrichment cultures, inoculated carriers, or other samples of microbiomes.

As used herein, the term “metagenomics” refers to the study of genetic material, “metagenomes”, recovered directly from environmental samples using nucleic acid sequencing of the metagenomes to produce profiles of microbial diversities present in the samples. Rapid advances in bioinformatics, DNA amplification techniques, and computational power and speed enabled the development and use of “shotgun” sequencing of whole genomes by randomly shearing DNA therein into many short sequences that are subsequently reconstructed, in reference to libraries of known sequences from cultured microorganisms, into consensus sequences. Analyses of the consensus sequences reveals genes that were present in the environmental samples, and thereby provide information about the types of microorganisms present and their relative abundance in the sample and also, about the types of metabolic processes that were occurring in the sample. Those skilled in this art will find a good comprehensive overview of the terms “microbiome”, “metagenome, and metagenomics” in Microbiome (2020) 8: 103 (Berg et al., Microbiome definition re-visited: old concepts and new challenges).

As used herein, the term “16S analysis” refers to the use of 16S rRNA gene sequencing of microbiomes present in environmental samples, for identification of and taxonomic grouping of the bacterial species present in the samples.

As used herein, the term “inoculation” refers to combining and culturing an enriched hydrocarbon-degrading microbiome with a selected carrier to produce agglomerated structures comprising mixtures of the hydrocarbon-degrading microbiome bound to the selected carrier as biofilms. The agglomerated structures are referred to herein as “aggregates”.

As used herein, the term “carrier” refers to a substrate that is suitable for combination and incubation with an enriched microbiome to thereby produce the agglomerated aggregates.

As used herein, the term “active biological countermeasures” refers to a composition comprising aggregates of an enriched hydrocarbon-degrading microbiome bound to a selected carrier as biofilms produced by microbial species comprising the enriched microbiome. It is to be noted that the term “active biological countermeasures” may be substituted herein by its acronym “ABCM”. It is to be noted that an ABCM composition is a dynamic composition wherein the numbers of each of the plurality of microbial species present in the enriched hydrocarbon-degrading microbiome will continually increase and/or decrease during the period of time between when the ABCM composition was produced and when it is deployed into a marine oil spill.

As used herein, the term “copepod” refers to a planktonic group of small crustaceans that are commonly present throughout seawater columns. The copepod life cycle comprises eggs, larvae, juveniles, and adults. Planktonic copepod species vary considerably in size but can typically be 1-2 mm long with teardrop shaped bodies and large antennae. Many planktonic copepods are mobile and capable of extremely fast movements.

As used herein, the term “surfactants” refer to amphiphilic compounds that can reduce surface and interfacial tension between immiscible fluids, for example oil and water, by accumulating at the interfaces between immiscible fluids to increase the solubility and mobility of hydrophobic or insoluble organic compound within one or both immiscible fluids. Suitable surfactants may be nonionic surfactants, anionic surfactants, cationic surfactants, and amphoteric surfactants. Non-limiting examples of nonionic surfactants include fatty acid esters and ethoxylated fatty acid esters. A non-limiting example of an anionic surfactant is sodium alkyl sulfosuccinate.

As used herein, the term “dispersants” refer to chemical mixtures of surface-active substances that are suitable for addition to oils spills for the purposes of colloiding and/or accelerating and/or improving the separation of oil particles and to prevent clumping of the oil particles. Non-limiting examples of commonly used dispersants include BIODISPERS° (BIODISPERS is a registered trademark of Petrotech America Corp., Cambridge, MA, US), COREXIT® (COREXIT is a registered trademark of Exxon Corp., Irving, TX, USA), DISPERSIT® SPC 1000 (DISPERSIT is a registered trademark of U.S. Polychemical Holding Corp., Chestnut Ridge, NY, US), NOKOMIS® 3-AA and 3-F4 (NOKOMIS is a registered trademark of Mar-Len Supply Inc., Hayward, CA, US), SEA BRAT® 4 (SEA BRAT is a registered trademark of Alabaster Corp., Pasadena, TX, US), among others. It should be noted that while dispersants are a form of surfactant, not all surfactants are dispersants.

As used herein, the term “practical salinity units” (PSU) refers to a unit of measurement based on the properties of seawater conductivity. PSU is defined in terms of the ratio K₁₅ of the electrical conductivity of a seawater sample at a temperature of 15° C. and a pressure of one standard atmosphere, in reference to that of a potassium chloride (KCl) solution, in which the mass fraction of KCl is 32.4356×10⁻³ at the same temperature and pressure.

As used herein, the term “about” refers to an approximately +/−10% variation from a given value. It is to be understood that such a variation is always included in any given value provided herein, whether or not it is specifically referred to.

It should be understood that the compositions and methods are described in terms of “comprising,” “containing,” or “including” various components or steps, the compositions and methods can also “consist essentially of or “consist of the various components and steps. Moreover, the indefinite articles “a” or “an,” as used in the claims, are defined herein to mean one or more than one of the elements that it introduces.

The embodiments according to the present disclosure generally relate to compositions for deployment into marine oil spills for rapid degradation of the hydrocarbon components of the oil spills to thereby ameliorate the toxicities and environmental pollution issues associated with the oil spills. The compositions are configured to preferentially and rapidly degrade hydrocarbon components comprising crude oil, marine fuels, bunker fuels, diesel fuels, and kerosene among others. According to some aspects, the compositions disclosed herein may be configured to preferentially degrade C₅₋₁₀ hydrocarbons and/or C₁₀₋₂₀ hydrocarbons and/or C₂₀₋₃₀ hydrocarbons and/or C₁₅₋₃₅ hydrocarbons and/or C₃₀₋₄₀ hydrocarbons and/or C₃₅₋₄₀ hydrocarbons and therebetween. Some of the compositions disclosed herein may be particularly suitable for deployment into small oil spills occurring in marine ocean environments. Some of the compositions disclosed herein may be particularly suitable for deployment into medium-size oil spills occurring in marine ocean environments. Some of the compositions disclosed herein may be particularly suitable for deployment into small and/or medium-size oil spills occurring in cold marine ocean environments wherein the seawater temperatures are in a range of about 1° C. to about 5° C.

Other embodiments according to the present disclosure generally relate to compositions for deployment into chronically long-term polluted marine water sites such as harbours, offshore mooring locations, channels connecting harbours and/or offshore mooring locations with open seas, and the like, for degradation of and amelioration of C₅₋₄₀ hydrocarbon components present in the long-term polluted seawater. Such compositions may be configured for regular periodic deployment into the long-term polluted marine water sites over extended periods of time to remediate the polluted marine water sites by sustained lowering of the C₅₋₄₀ hydrocarbon components present in the polluted seawater. According to some aspects, the compositions disclosed herein may be configured to preferentially degrade C₅₋₁₀ hydrocarbons and/or C₁₀₋₂₀ hydrocarbons and/or C₂₀₋₃₀ hydrocarbons and/or C₁₅₋₃₅ hydrocarbons and/or C₃₀₋₄₀ hydrocarbons and/or C₃₅₋₄₀ hydrocarbons and therebetween. Some of the compositions disclosed herein may be particularly suitable for deployment throughout multiple locations within a marine harbour. Some of the compositions disclosed herein may be particularly suitable for deployment throughout multiple locations within an offshore mooring location. Some of the compositions disclosed herein may be particularly suitable for deployment within marine harbours and/or offshore mooring sites wherein the seawater temperatures are in the range of about 1° C. to about 5° C.

Some embodiments according to the present disclosure are related to methods for preparing the compositions disclosed herein.

Some embodiments according to the present disclosure are related to systems for rapid delivery and deployment of the compositions disclosed herein, into marine oil spills.

According to one example embodiment, a method for preparing an ABCM composition configured for deployment into a marine oil spill, may comprise the steps of:
1. collecting a seawater sample from a water column located in a marine harbour or an offshore mooring site;
2. enriching a microbiome present in the seawater sample by culturing in a selected liquid medium with minimal or alternatively without any carbon nutrients but which is supplemented with one or more selected hydrocarbon-containing components for a period of at least 7 days or more, for example 10 days or more, 14 days or more, 18 days or more, 21 days or more, or longer, to thereby produce an enriched microbiome;
3. combining the enriched microbiome with a selected carrier and incubating the combined enriched microbiome and carrier for at least 18 h while gently commingling the enriched microbiome and carrier, for example at a rate selected from a range of about 0.5 RPM to about 30 RPM, to produce the composition.

It is to be noted that the composition may comprise agglomerated microbial components of the enriched microbiome and carrier particles that are loosely bound together by biofilms formed and secreted by the microbial components of the enriched microbiome. It is to be noted that the agglomerated structures are also referred to herein as “aggregates”. In other words, the present composition may comprise aggregates that include microbial components of the enriched microbiome attached to carrier particles by biofilms secreted by the microbial components.

According to one aspect, a suitable medium for culturing and enriching a microbiome present in a seawater sample may be a selected suitable microbial culture liquid medium known to those skilled in this art, from which the carbon source has been omitted and substituted for with a selected C₅₋₄₀ hydrocarbon-containing component. Suitable microbial culture liquid media that may be used include minimal media known in the art, from which carbon sources are omitted.

According to another aspect, a suitable medium for culturing and enriching a microbiome present in a seawater sample may be one or more of a carbon-starved minimal medium such as Bushnell Haas nutrient broth, Väätänen Nine-Salt Solution (VNSS), marine broth, Luria-Bertani marine broth, 2% NaCl Mueller-Hinton broth, Zobell marine broth, and the like.

According to another aspect, a suitable C₅₋₄₀ hydrocarbon-containing component for supplementing a selected culture medium may be any one of a light crude oil, a heavy crude oil, a marine fuel, a bunker fuel, a diesel fuel, kerosene, and the like. Also suitable for supplementing a selected culture medium are individual C₅₋₄₀ hydrocarbons such as benzene, toluene, ethylbenzene, naphthalene, xylene, among others.

According to another aspect, the enriching step for culturing hydrocarbon-degrading microbial populations present in the microbiome of a collected seawater sample may be done at a culturing temperature selected from a range of about 1° C. to about 25° C. A particularly suitable temperature range for culturing hydrocarbon-degrading microbial populations present in the microbiome of a collected seawater sample may be from a range of about 5° C. to about 10° C.

According to another aspect, enriched hydrocarbon-degrading microbiomes produced according to above method steps 1 and 2 may comprise a plurality of microorganisms such as Acanthopleuribacter spp., Acinetobactor spp., Alcanivorax spp., Alteromonadaceae spp., Cycloclasticus spp., Flavobacterium spp., Hyphomonas spp., Marinobacter spp., Porticoccaeae spp., Pseudomonas spp., Rhodobacteraceae spp., Sphingopyxis spp., Sphingobium spp., Xanthobacter spp., among others. Most of the enriched organisms may be hydrocarbon degraders, while others may be involved in biofilm formation.

According to another aspect, suitable carriers for combining with an enriched microbiome may be particulate crustacean shells, particulate exoskeletons or shells of marine molluscs, particulate microalgae, particulate macroalgae, clays, zeolites, the like and combinations thereof. The particulate carriers may be produced by pulverizing or grinding. Suitable particle sizes for the carriers are in a range of about 50 μm to about 5 mm and therebetween. Particularly suitable carrier particle sizes are from a range of about 200 μm to about 1 mm and therebetween.

According to another aspect, suitable carriers for combining with an enriched microbiome may be crustacean carcasses, eggs, larvae, juveniles, and combinations thereof. The crustacean eggs and juveniles may be living or not. Suitable crustaceans may be krill, copepods, and the like. Particularly suitable are planktonic copepod carcasses, eggs, juveniles, and combinations thereof. It is to be noted that combining and incubating enriched microbiomes with live crustacean eggs and/or juveniles as the carrier, may result in germination of the crustacean eggs during the incubation period thereby producing an ABCM composition that, upon delivery onto and into an oil spill, will be more rapidly dispersed throughout the spill by the swimming movements of the enriched microbiome-inoculated crustacean larvae and/or juveniles.

According to another embodiment of the present disclosure, the ABCM compositions produced according to methods disclosed herein, may additionally be provided with one or more selected C₅₋₄₀ hydrocarbons.

According to another embodiment of the present disclosure, the ABCM compositions produced according to methods disclosed herein, may additionally be provided with one or more of surfactants, emulsifiers, enzymes, dispersants, and the like.

According to another embodiment of the present disclosure, the ABCM compositions produced according to methods disclosed may be delivered onto and into a small or a medium-size marine oil spill by pressurized spraying through a nozzle. The ABCM compositions may be delivered to the marine oil spill site in barrels and/or bulk tanks loaded onto/into a vessel and then deployed into spill by pressurized spraying. Alternatively, the ABCM compositions may be delivered to a marine oil spill site in barrels and/or bulk tanks loaded onto/into aircraft from which, the ABCM compositions are delivered into the spill by dropping, i.e., dispensing the ABCM compositions from the barrels and/or bulk tanks from an aircraft in flight.

Another embodiment of the present disclosure relates to methods for rapid response to a small or a medium-size marine oil spill by preparing and delivering thereinto selected ABCM compositions.

The methods may comprise a step of collecting seawater samples from water columns in a plurality of locations throughout selected marine offshore locations that have experienced chronic contamination or “at risk” locations. “At risk” as used herein refers to an increased probability of a potential small or medium-size spill in a geographical marine area due to a high volume therethrough of ocean vessel traffic conveying light crude oil, heavy crude oil, marine fuel, bunker fuel, diesel fuel, kerosene, and the like. Thus, as defined herein most “at risk” locations will also have experienced chronic contamination.

The methods may comprise a step of dividing each of the collected seawater samples into subsamples, and then enriching each of the subsamples with a different selected hydrocarbon-containing oil product added to a selected liquid nutrient medium. For example, one subsample may be enriched with a selected light crude oil thereby producing a light crude oil-degrading microbiome, another subsample may be enriched with a selected marine fuel oil thereby producing a marine fuel oil-degrading microbiome, yet another subsample may be enriched with a selected diesel fuel thereby producing a diesel fuel-degrading microbiome, and so on. It is to be noted that suitable liquid nutrient media include one or more of Bushnell Haas nutrient broth, Väätänen Nine-Salt Solution (VNSS), marine broth, Luria-Bertani marine broth, 2% NaCl Mueller-Hinton broth, Zobell marine broth, and other suitable media known to those skilled in this art. Also suitable are liquid nutrient media known to those skilled in the art, from which the carbon sources have been omitted and substituted for with one or more selected C₅₋₄₀ hydrocarbon compounds. If so desired, the subsamples may be enriched in two or three or four or five or six or seven or eight or nine or ten or more different selected C₅₋₄₀ hydrocarbons

The methods may comprise a step of maintaining an enriched hydrocarbon-degrading microbiome by one of a continuous culture process or a batch culture process. If a continuous culture process is selected for maintaining an enriched hydrocarbon-degrading microbiome, then the selected nutrient medium and the selected hydrocarbon-containing oil product may be supplied to the enrichment culture vessel at selected constant rates while enriched microbiome is removed from the enrichment culture vessel at a rate equivalent to the input rates. If a batch culture is selected for maintaining an enriched hydrocarbon-degrading microbiome, then at a selected time wherein the enriched microbiome is in a steady state, the batch culture be separated into two or more portions wherein one of the portions is transferred to a fresh batch culture vessel containing therein the selected nutrient medium and the selected hydrocarbon-containing oil product for continued enrichment and maintenance of the hydrocarbon-degrading microbiome. If so desired, the two or three or four or five or six or seven or eight or nine or ten or more enriched hydrocarbon-degrading microbiomes may be maintained in two or three or four or five or six or seven or eight or nine or ten or more different nutrient media, each containing different selected C₅₋₄₀ hydrocarbons.

In the event of a small or a medium-size marine oil spill, the volumes of one or more maintained enriched hydrocarbon-degrading microbiome may be rapidly scaled up accelerated culturing in the selected nutrient medium amended with a selected hydrocarbon oil product. It is optional, if so desired, to collect water samples from within and about a marine oil spill location and then culture the water samples to facilitate the growth and enrichment of inherent microbiome populations for deployment into the oil spill. Methods of accelerating and increasing volumes are well known and include (i) transferring all of the separated enriched microbiome portions into fresh amended nutrient medium, and (ii) slightly increasing the temperature by several ° C. and increasing the mixing rates in the culture vessels.

The next step may involve combining the volumes of enriched hydrocarbon-degrading microbiomes with one or more selected carriers, and then gently mixing the combination at a rate from about 0.5 RPM to about 30 RPM for a selected time period to produce the ABCM compositions that are suitable for delivery into the oil spill. Suitable time periods may be 2 h, 4 h, 8 h, 12 h, 16 h, 20 h, 24 h, and therebetween. It is optional to select longer time periods if so desired, for example 36 h, 48 h, 60 h, or longer. The ABCM compositions will not remain static or constant after the formulation processes are completed but rather, the numbers of each individual hydrocarbon-degrading microbial specie present in the microbiome at the time of preparation of the ABCM composition, may subsequently increase and/or decrease over a period of time prior to deployment of the dynamic microbiome-based ABCM composition into a marine oil spill.

It is an optional opportunity during the maintenance of an enriched hydrocarbon-degrading microbiome culture, to combine the portions of enriched microbiome cultures harvested and removed during the maintenance operations, with a selected carrier and then gently mixing the combination at a rate from about 0.5 RPM to about 30 RPM for 24 h to produce the ABCM compositions that are suitable for routine regular delivery into selected locations of long-term hydrocarbon-polluted seawater in marine harbours and offshore mooring sites for remediation of the hydrocarbon pollution seawater.

### EXAMPLES

### Example 1

The first study assessed the potential for producing an oil-degrading composition for rapid deployment into and degradation of a marine oil spill. There were five parts to this study:
(i) collection of water samples from the epipelagic zones of water columns in hydrocarbon-polluted marine environments, with the expectation that those water samples will comprise oil-degrading microbiomes,
(ii) enriching the oil-degrading microbiomes by culturing in selected nutrient media supplemented with crude oil or diesel fuel,
(iii) combining the enriched oil-degrading microbiomes with a selected carrier and further incubating the microbiome-carrier mixture in selected nutrient media supplemented with crude oil or diesel fuel,
(iv) adding the enriched microbiome-carrier mixture to seawater into which crude oil or diesel fuel (i.e., hydrocarbons) was added, and
(v) monitoring growth of the microbiome on the hydrocarbons over selected periods of time.

### Water Sample Collection

A number of experiments were performed with a hydrocarbon-polluted seawater sample collected from Copenhagen Harbour (55°41′42.4″N 12°36′00.7″E), Denmark.

### Enrichment of Naturally Occurring Oil-Degrading Microbiomes

Subsamples of Copenhagen Harbour seawater were dispensed into 15-mL conical centrifuge tubes (replicates of 5-mL subsamples; 7.5-mL subsamples; 10-mL subsamples). Then, to each tube was added Bushnell Haas nutrient broth (VWR International, Mississauga, ON, CA) at a ratio of 0.5 mg/mL seawater, and 0.025 ml of a petroleum diesel fuel (EN 590 standard) (10 μL fuel/5 mL seawater). The subsamples were incubated at ambient temperatures on a shaker table for two weeks. Growth of microbial populations comprising the microbiomes was monitored by measuring the increasing optical densities (OD) of the incubated subsamples.

### Carriers

Three carriers were assessed in this study: (i) copepod carcasses, (ii) copepod eggs, and (iii) microalgae.

Adult copepods are commonly harvested by fishing boats and are processed by extraction of their fatty acids for use as supplements for human and animal nutrition. Copepod carcasses are a byproduct of this process and are commercially available for use as fish food. The copepod carcasses used in this study were obtained from Calanus AS (Norway). The Calanus AS copepod carcasses were filtered to remove particles that were smaller than 200 μm.

Copepods have the ability to go into dormancy in response to deteriorating environmental conditions whereby they form quiescent diapaused eggs. Consequently, quiescent copepod eggs can be stored for extended periods of time (e.g., at low temperatures). Copepod eggs can be manipulated to germinate and become physiologically active by providing suitable culture conditions (e.g., increasing temperature and providing nutrients. The quiescent copepod eggs used in this study were of the calanoid copepod Acartia tonsa supplied by Calanus AS.

The marine microalgae used in this study was Tetraselmis spp. supplied by the Geological Survey of Denmark and Greenland Institute (GEUS).

The enriched oil-degrading microbiomes were combined with one of: (i) the copepod carcasses at a density of 0.1 mg carcasses per mL microbiome, (ii) copepod eggs at a density of 1000 eggs per mL microbiome, and (iii) and microalgae at an OD of 0.025 per mL microbiome.

The mixtures of enriched oil-degrading microbiomes and carriers were then attached to a plankton wheel and incubated for 24 h at ambient temperatures at 3 RPM. It was noted that all of the enriched microbiome and carrier combinations formed agglomerations referred to herein as “aggregates”.

### Crude Oil Degradation by the Enriched Microbiome-Carrier Mixtures

Three separate experiments were conducted to assess the degradation of crude oil added to seawater samples.

The first experiment assessed crude oil degradation by a mixture of enriched microbiome combined and incubated with copepod carcasses, with the following treatments:
1. seawater control
2. seawater amended with crude oil
3. seawater amended with crude oil to which empty un-inoculated copepod carcasses were added
4. seawater amended with crude oil to which an aliquot of the enriched microbiome was added
5. seawater amended with crude oil to which the enriched microbiome—copepod carcass mixture was added

The treatments were loaded onto a shaker table that was installed inside a sealed incubation chamber where the samples were kept in semi-darkness and a temperature-controlled environment at 20° C. The samples were removed from the mixing table at 24 h intervals for measurement of their OD at 550 nm with a PALINTEST® 7100 Photometer (PALINTEST is a registered trademark of Palintest Ltd., Gateshead, UK).

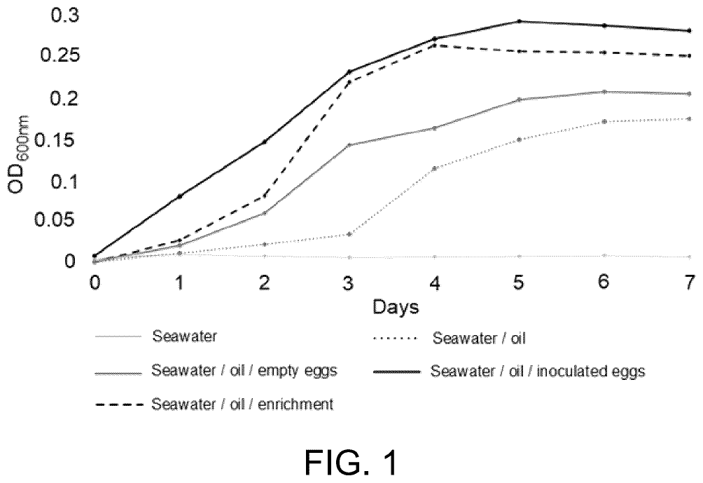

The data in FIG. 1 show that microbial growth occurred most rapidly and to a greater extent in treatment 5 (seawater amended with crude oil to which the enriched microbiome—copepod carcass mixture was added) followed by treatment 4 (seawater amended with crude oil to which an aliquot of the enriched microbiome was added). It was noted that some microbial growth occurred in treatments 2 and 3 indicating that the seawater used in this experiment comprised an innate population of oil-degrading microorganisms.

The second experiment assessed crude oil degradation by a mixture of copepod eggs that were inoculated and incubated with an enriched microbiome, with the following treatments:
1. seawater control
2. seawater amended with crude oil
3. seawater amended with crude oil to which copepod eggs were added
4. seawater amended with crude oil to which an aliquot of the enriched microbiome was added
5. seawater amended with crude oil to which the enriched microbiome—copepod eggs mixture was added

After the copepod eggs and enriched microbiome were combined and mixed together for 24 h as described in the “Carriers” section above, a portion of the mixture was removed and inspected microscopically. Copepod eggs typically hatch within 24-72 hours after transfer from cold anoxic conditions to oxygen saturated water at room temperature. After the 24-h mixing period, it was observed that some of the quiescent copepod eggs had hatched in the incubation period and live nauplii (the initial life stage of copepods) were swimming throughout the sample. Also present in the sample were unhatched eggs and empty eggshells.

The treatments were loaded onto a shaker table that was installed inside a sealed incubation chamber where the samples were kept in semi-darkness and temperature-controlled environment at 20° C. The samples were removed from the mixing table at 24 h intervals for measurement of their optical densities (OD) at 550 nm with a PALINTEST® 7100 Photometer.

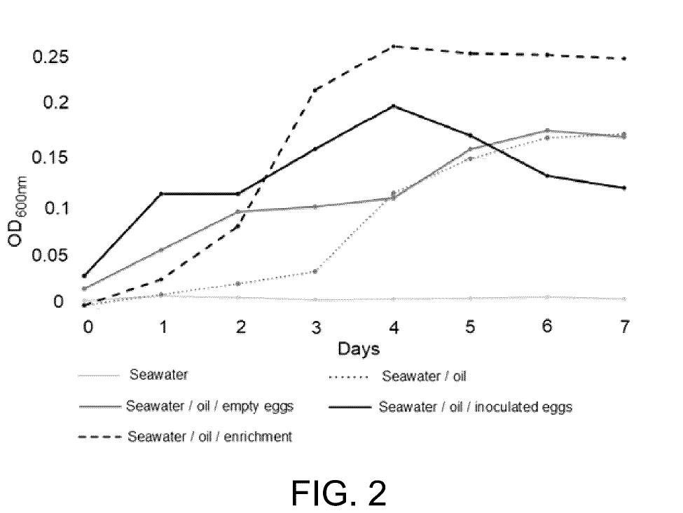

The data in FIG. 2 show that microbial growth occurred most rapidly and to a greater extent in treatment 5 (seawater amended with crude oil to which the enriched microbiome—copepod carcass mixture was added) followed by treatment 4 (seawater amended with crude oil to which an aliquot of the enriched microbiome was added). It was noted that some microbial growth occurred in treatments 2 and 3 indicating that the seawater used in this experiment comprised an innate population of oil-degrading microorganisms.

The third experiment assessed crude oil degradation by a mixture of enriched microbiome combined and incubated with microalgae, with the following treatments:
1. seawater control
2. seawater amended with crude oil
3. seawater amended with crude oil to which microalgae were added
4. seawater amended with crude oil to which an aliquot of the enriched microbiome was added
5. seawater amended with crude oil to which the enriched microbiome—microalgae mixture was added

The treatments were loaded into a mixing table that was installed inside a sealed incubation chamber where the samples were kept in semi-darkness and temperature-controlled environment at 20° C. The samples were removed from the mixing table at 24 h intervals for measurement of their optical densities (OD) at 550 nm with a PALINTEST® 7100 Photometer.

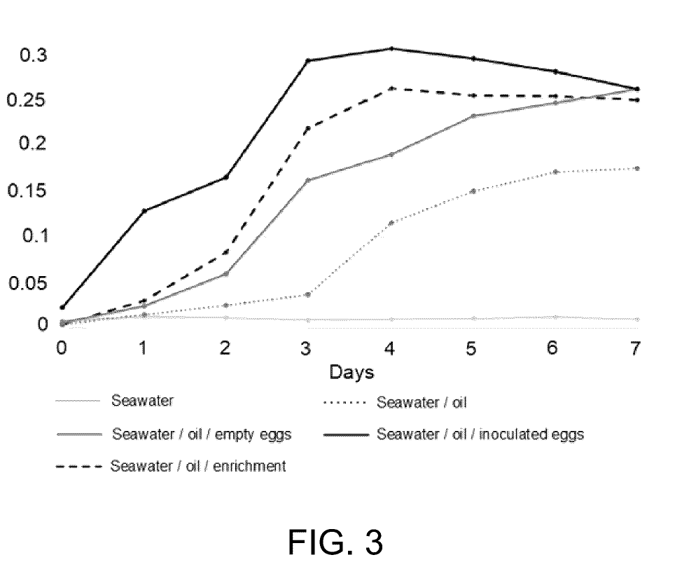

The data in FIG. 3 show that microbial growth occurred most rapidly and to a greater extent in treatment 5 (seawater amended with crude oil to which the enriched microbiome-microalgae mixture was added) followed by treatment 4 (seawater amended with crude oil to which an aliquot of the enriched microbiome was added). It was noted that some microbial growth occurred in treatments 2 and 3 indicating that the seawater used in this experiment comprised an innate population of oil-degrading microorganisms.

### SUMMARY

Each of the three carriers assessed in this study (i) copepod carcasses, (ii) copepod eggs, and (iii) microalgae, agglomerated with enriched oil-degrading microbiomes within 24 h of incubation in enriched seawater under gentle mixing conditions (i.e., 3 RPM) to form aggregates that presumably were held together by biofilms that were secreted by microbial species comprising the enriched microbiomes.

Addition of the enriched microbiome-carrier aggregates to seawater amended with crude oil resulted in rapid onset of microbial growth.

### Example 2

This study assessed the scalability of ABCM compositions and their degradation of selected crude oil components in North Atlantic Ocean seawater.

Studies were performed in closed systems (200-mL bottles) using 150-mL volumes of marine water amended with one of benzene, toluene, ethylbenzene, naphthalene, o-xylene, and a mixture of m-xylene and p-xylene, following the methods described in Example 1. The controls were unamended marine water.

Three treatments were assessed with each of the six hydrocarbons. Treatment 1 was a control consisting of seawater only. Treatment 2 was seawater to which was added an enriched hydrocarbon-degrading microbiome. Treatment 3 was seawater to which was added an ABCM composition comprising copepod carcasses inoculated with and incubated with an enriched hydrocarbon-degrading microbiome for 24 h.

Naturally occurring hydrocarbon-degrading microbiomes present in a water sample collected from a polluted seawater column were enriched following the process outlined in Example 1. After 35 days of enrichment, the enriched microbiome was combined with copepod carcasses used obtained from Calanus AS (Norway). The Calanus AS copepod carcasses were sieved to remove particles that were smaller than 200 pm prior to inoculation with the enriched microbiome. The combined copepod carcasses and enriched microbiome were attached to a plankton wheel and then incubated for 24 h at ambient temperatures under gentle mixing (3 RPM) to produce the ABCM composition.

The hydrocarbon-degradation performances of the ABCM composition and the enriched microbiome were assessed by (i) using optical density to measure microbial growth that occurred during the experimental time period, and (ii) detecting the amounts of benzene, toluene, ethylbenzene, naphthalene, o-xylene, and a mixture of m-xylene and p-xylene that remained in the samples at the conclusion of the experiment time period, with gas chromatography using a flame-ionization detector (GC-FID).

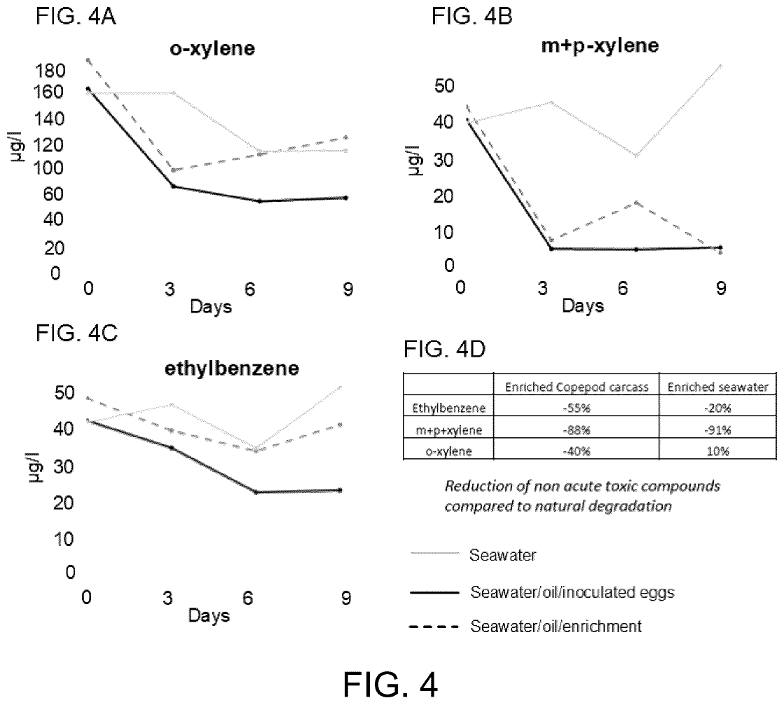

FIG. 4 shows charts illustrating the effects of the ABCM treatment and the enriched microbiome on the degradation of o-xylene (FIG. 4A), a mixture of m-xylene and p-xylene (FIG. 4B), and ethylbenzene (FIG. 4C) in seawater samples. The Table in FIG. 4D shows that the ABCM treatment reduced the levels of (i) m-xylene and p-xylene by 88% compared to natural degradation in the seawater control, (ii) ethylbenzene by 55% compared to natural degradation in the seawater control, and (iii) o-xylene by 40% compared to natural degradation in the seawater control. Additionally, the Table in FIG. 4D shows that the enriched hydrocarbon-degrading microbiome reduced the levels of (iv) m-xylene and p-xylene by 91% compared to natural degradation in the seawater control, (v) ethylbenzene by 20% compared to natural degradation in the seawater control, and (vi) o-xylene by 10% compared to natural degradation in the seawater control.

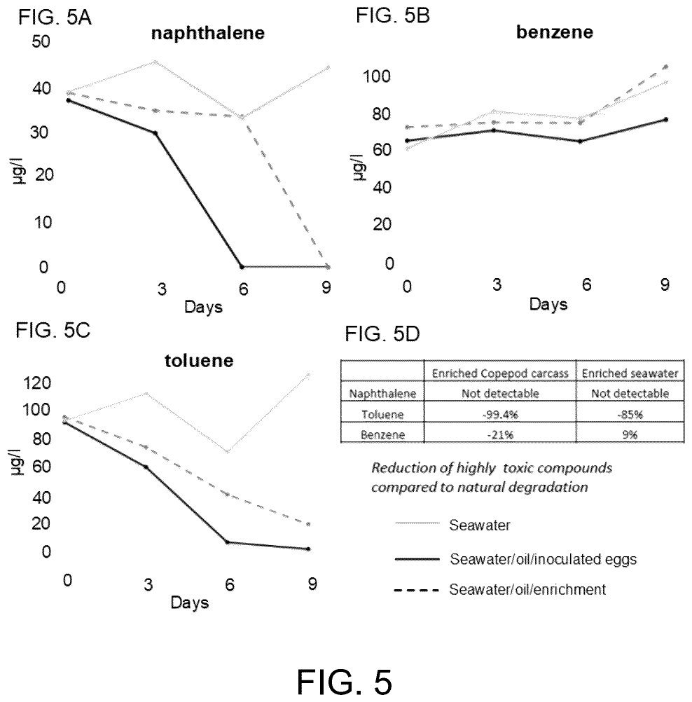

FIG. 5 shows charts illustrating the effects of the ABCM treatment and the enriched microbiome on the degradation of naphthalene (FIG. 5A), benzene (FIG. 5B), and toluene (FIG. 4C) in seawater samples. FIG. 5A and the Table in FIG. 5D show the ABCM treatment completely degraded naphthalene in 6 days while the enriched microbiome degraded naphthalene in 9 days, while there was no change in the seawater control. FIG. 5B and the Table in FIG. 5D indicate that at the end of the 9-day trial period, the benzene level was decreased by 21% by the ABCM treatment and by 9% by the enriched microbiome. FIG. 5C and the Table in FIG. 5D show the ABCM treatment reduced the toluene level by 99.4% by the end of the 9-day trial period days, while the enriched microbiome reduced the toluene level by 85% over the 9 days, while there was no change in the seawater control.

These data show that the ABCM compositions and the enriched microbiomes assessed in this study, significantly increased the rates of degradation and extent of degradation of benzene, toluene, ethylbenzene, naphthalene, o-xylene, m-xylene, and p-xylene, added to seawater samples, in reference to control seawater samples.

### Example 3

This study assessed the scalability of performance of ABCM compositions to degrade crude oil in larger volumes of marine water collected from the North Sea region of the Atlantic Ocean and having salinity of 30 PSU or greater. Issues considered in this study included determining: (i) the effects of ABCM compositions when applied to simulated oil slicks on the surfaces of seawater in 200-L barrels, and (ii) determining the effects of larger-volume open systems, gas exchange, evaporation, and water-column mixing on the ABCM performance.

Seawater used for enrichment of a hydrocarbon-degrading microbial community therein, was collected (100 L) from Hirtshals Harbour in Denmark and subsampled into 10 L volumes (57°35.541′N 9°57.698′E). Hirtshals Harbour has been and remains chronically polluted with hydrocarbons. The seawater sample was filtered through a 150-μm mesh screen and then distributed into four individual 5-L blue cap bottles, with approximately 2.5 L of filtered seawater in each bottle. To enrich the growth of oil-degrading microorganisms in the filtered seawater, Nuuk diesel (a commercial fuel-grade diesel fuel) was added to each bottle at a ratio of 0.5 mL per litre of seawater (1.25 g diesel per 2.5 L seawater sample). To ensure a high growth of microorganisms, Bushnell Haas nutrient broth (VWR International, Mississauga, ON, CA) was added at a ratio of 0.5 g per litre of seawater (1.25 g BH broth per 2.5 L seawater sample). Additional diesel and nutrients were added and the headspace air was renewed from each 5-L bottle at 2-day intervals for a 35-day enrichment period.

### Preparation of ABCM Carriers

Six treatments were assessed in this study as shown in Table 1. Three different carriers were assessed in three of the treatments: (1) carcasses of the marine zooplankton copepods (in ground form, produced at industrial scale as aquarium food), (2) marine microalgae (Rhodomonas salina), and (3) macroalgae (kelp, in ground form, produced at industrial scale as garden and orchard fertilizer).

Each of the four 250-L enriched microbial community volumes was designated as one of Treatments 1, 2, 3, and 4. Treatment 4 did not receive a carrier (Table 1). Each of the four treatment volumes was divided into 4 aliquots with each aliquot dispensed into a 5-L bottle, after which, additional seawater was added to bring the total volume in each bottle up to 4.6 L (Table 1). The 16 bottles were then equidistantly spaced around a plankton wheel and incubated for 24 hr at ambient temperature (about 21° C.) under slow rotation (3 RPM) of the plankton wheel. It was noted that aggregate formation became visible in the enriched copepod treatment 1 within the first 15 min of the 24 hr incubation period.

TABLE 1

| Treatment | Carrier | | Enrichment | Seawater | Total Volume |
|---|---|---|---|---|---|
| 1. Copepod carcass & enrichment | 1.5 | g | 2 L | 2.6 L | 4.6 L |
| 2. Microalgae (R. salina) & enrichment | 900 | mL | 2 L | 1.7 L | 4.6 L |
| 3. Macroalgae (dired kelp) & enrichment | 1.5 | g | 2 L | 2.6 L | 4.6 L |
| 4. Enrichment only (no carrier) | — | | 2 L | 2.6 L | 4.6 L |
| 5. Copepod carcass (no enrichment) | 1.5 | g | — | 1.0 L | 1.0 L |
| 6. Seawater control | — | | — | — | — |

NOTES
The copepod carcasses without enrichment were not put on a plankton wheel, but were diluted in a 1-L of seawater for 24 hr prior to use.
The microalgae were at a concentration of ~3 × 10⁵ cells mL⁻¹.

### Preparation, Inoculation, and Monitoring of the Test Barrels

Seawater (1,200 L) was collected from Esbjerg Harbour, Denmark and transported to the study site. Esbjerg Harbour has been and remains chronically polluted with hydrocarbons. 180 L of seawater was transferred into each of six 200-L test barrels. The seawater was stored in darkness at ambient temperatures with 100 L/h aeration until the treatments were added and the study period commenced.

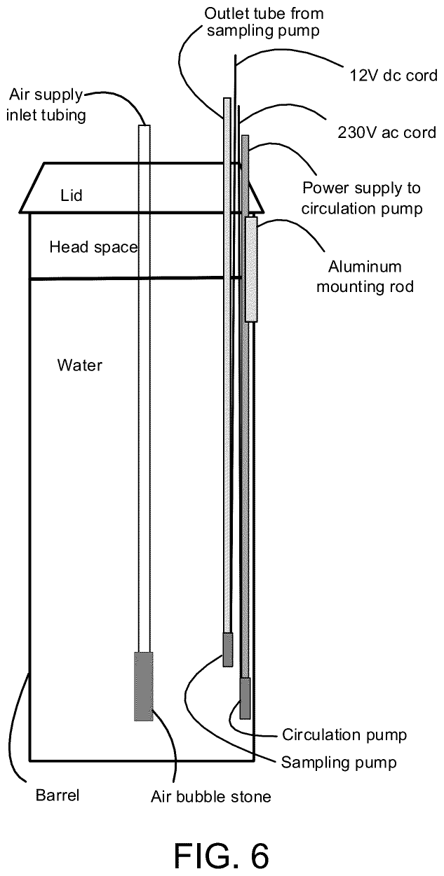

The design and set up of the six 200-L barrels are illustrated in FIG. 6. Each barrel was provided with a 230V 100 l/h aquarium circulation pump mounted about 25 cm above the bottom of the barrel to provide horizontal circulation of the seawater. A 12V DC groundwater sampling pump equipped with a 10 mm polypropylene tubing for sampling, was mounted about 50 cm above the bottom. An air stone connected to a 4 mm silicone tube, was inserted through the center of the lid to a depth of about 5 cm above the bottom to provide about 100 l/h of air for the purpose of providing oxygen and vertical water circulation throughout the seawater. Sampling tubes and electrical cords connected to electrodes were passed through a 1×4 cm hole at the periphery of the lid to enable the lid to remain fixed-in-place during the duration of the study period to prevent photodegradation and particulate contamination of the barrels. The electrodes were selected to measure (1) water temperature, (ii) oxygen saturation, (iii) eH, (iv) pH, and (v) conductivity.

To each of the six 200-L barrels was added 30 g of Bushnell Haas nutrient broth after which, the pumps and air stones were turned on to provide mixing of the nutrient broth throughout the 180 L of seawater. Then, the barrels received the treatments as follows:
Barrel A: treatment 3 (trt3; ABCM kelp carrier),
Barrel B: treatment 2 (trt2; ABCM microalgae carrier),
Barrel C: treatment 4 (trt4; enriched microbiome only—no carrier),
Barrel “D”: treatment 6 (trt6; seawater control),
Barrel “E”: treatment 1 (trt1; ABCM copepod carcasses carrier),
Barrel “F”: treatment 5 (trt5; copepod carcasses).

Then, 180 mL of oil were added to the top of each barrel to simulate an oil slick and the barrels were sealed with constant supply of oxygen and vertical circulation of the seawater. The barrels were maintained at ambient temperatures (about 22° C. to about 24.5° C.) for the duration of the study.

Data collected by the electrodes were recorded: (i) just prior to the addition of the microbial treatments and crude oil slick, (ii) at 8-h intervals for 48 h after addition of the treatments, (iii) then daily (i.e., every 24 h) for 7 days (i.e., through 9 days after the treatments and oil were added to the barrels), and (iv) then once every 7 days for the duration of the 5-wk study period. It was also noted that the oil slicks had disappeared from the tops of barrels that received the ABCM treatments, that is, barrels A, B, C, and E.

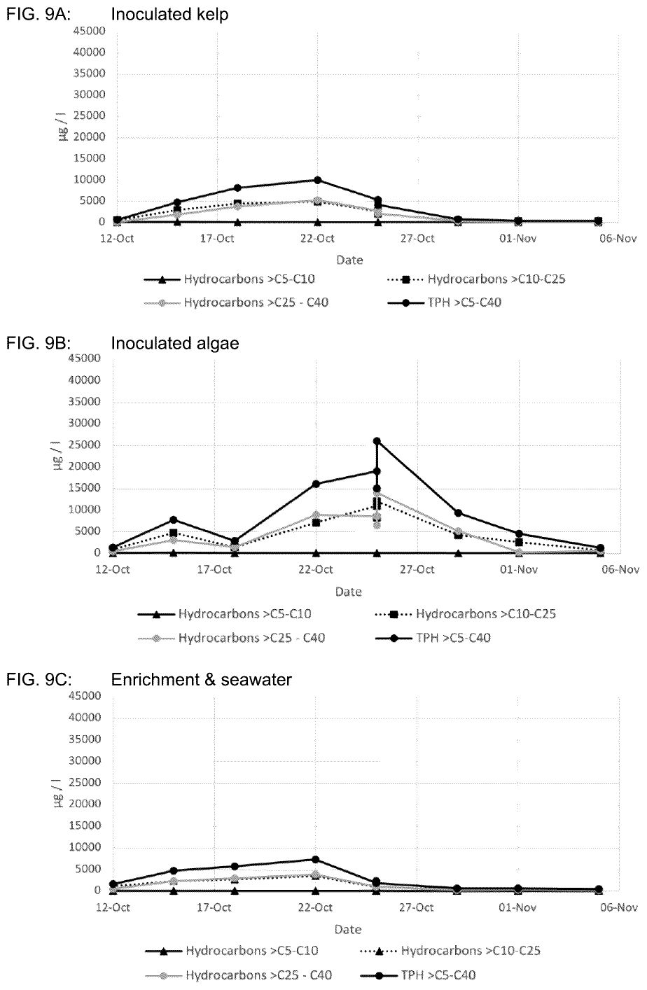

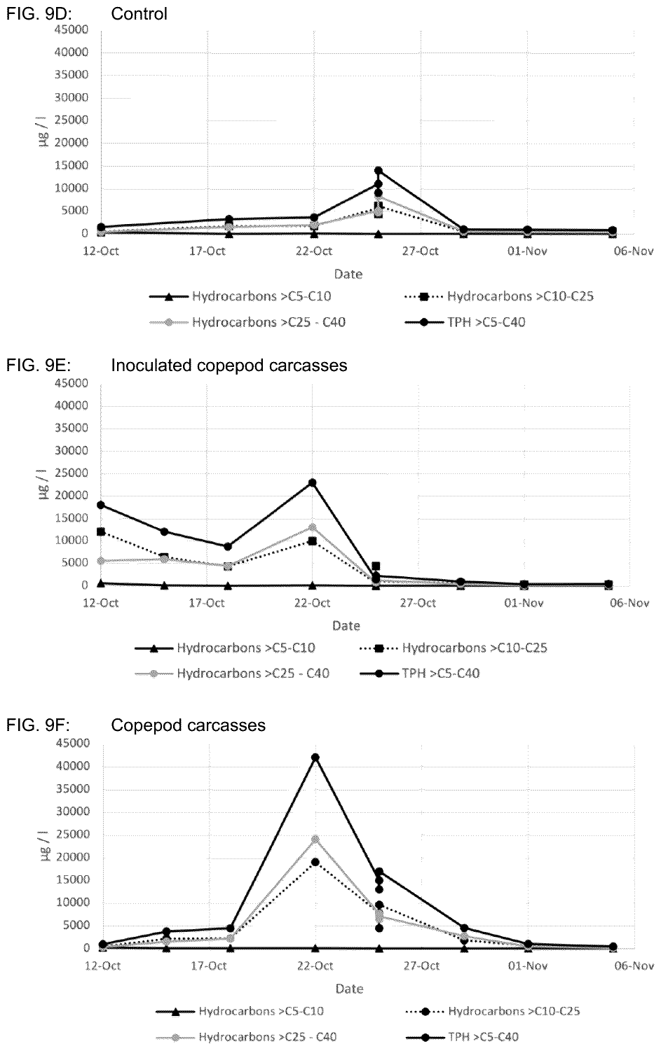

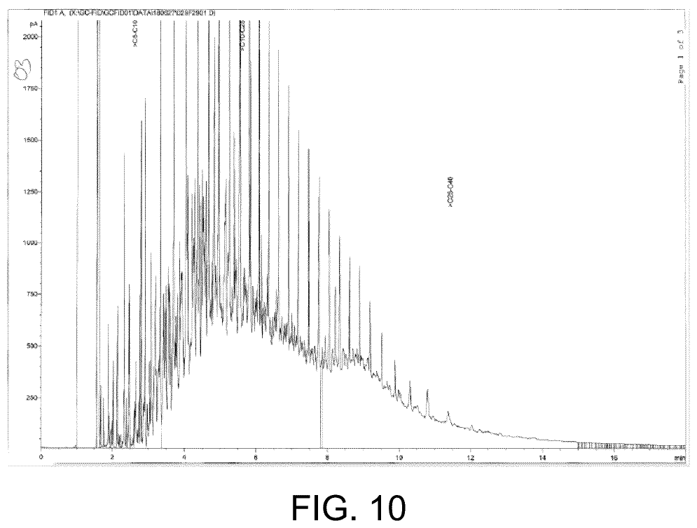

Water samples were collected from each of the six barrels for analyses of their hydrocarbon contents at: (i) 8 h, 16 h, and 24 h after addition of the treatments, (ii) then at 2 days, 5 days, and 9 days, and (iii) then once every 7 days for the duration of the study period. Each of the 150 mL samples was analysed by GC-FID to detect and quantify their content of (i) total petroleum hydrocarbons (TPH), (ii) C₂₅₋₄₀ hydrocarbons, (iii) C₁₀₋₂₅ hydrocarbons, and (iv) C₅₋₁₀ hydrocarbons. The results are shown in FIGS. 9A-9F. The surface oil slicks had disappeared from the surface of the test barrels and the detectable levels of TPH were minimal.

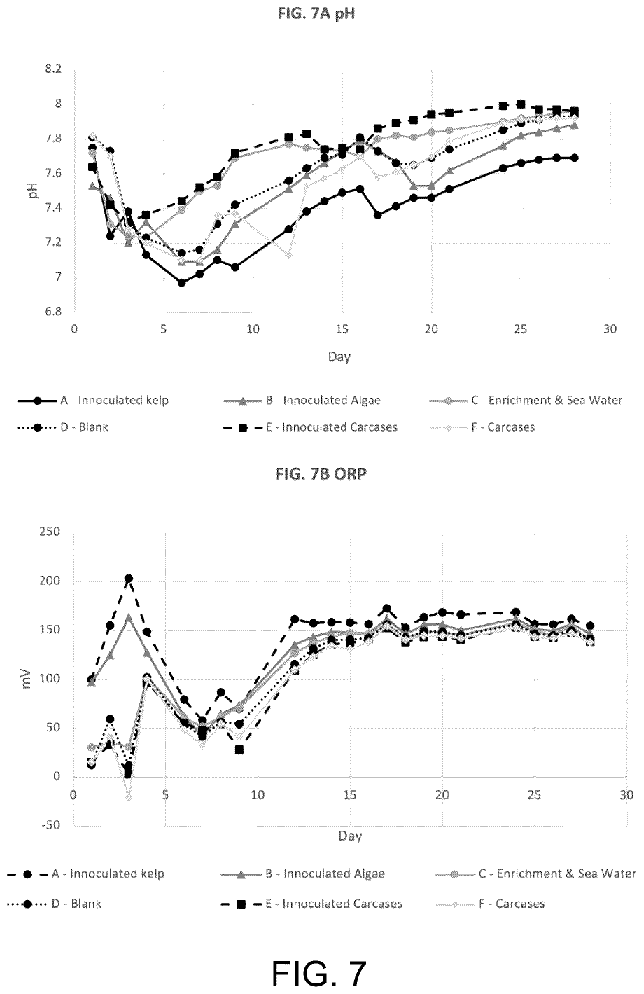

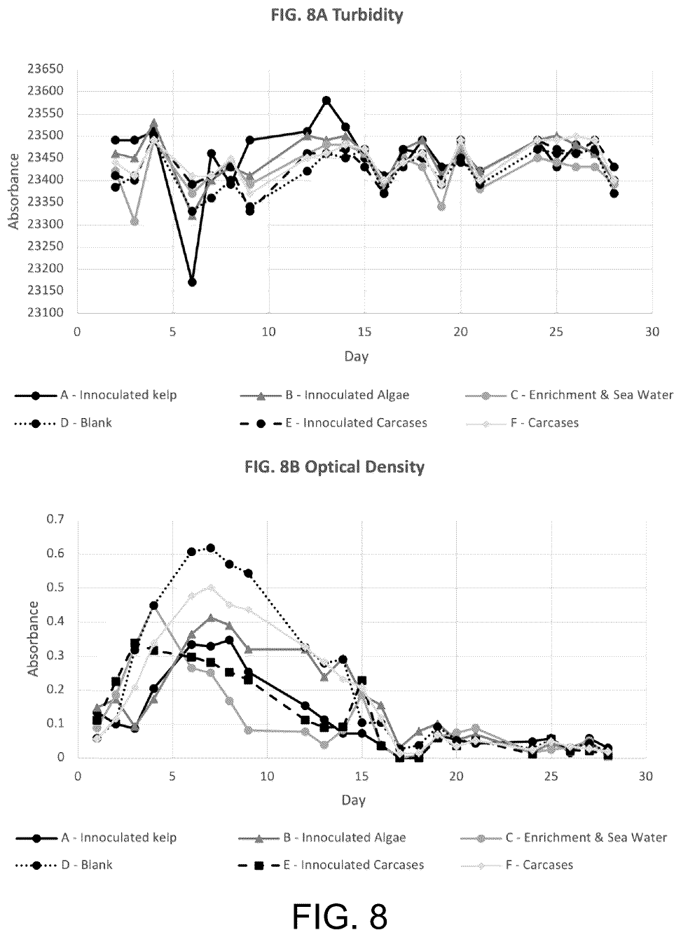

The pH, eH (oxidative reduction potential), and turbidity measurements (OD600 nm) show some effects of the ABCM treatments during the first 10 days after the study commenced, but were about the same in all of the barrel treatments by day 26 (FIGS. 7A, 7B, 8A). Optical density measurements (600 nm), indicative of microbial growth, increased and peaked in all treatments about 5-7 days after the study commenced and then returned to and remained at baseline levels by 18 days and thereafter (FIG. 8B).

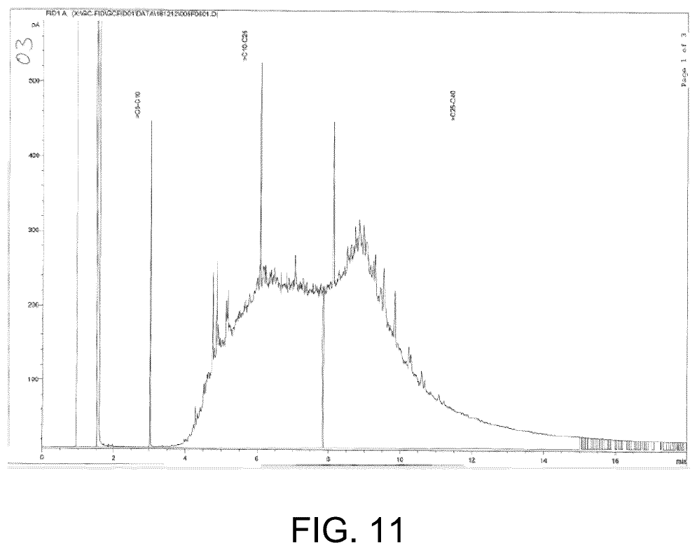

GC-FID analyses of water samples collected from the test barrels 1 day after the study commenced, showed the presence of high concentrations of dissolved alkanes (FIG. 10). The absence of alkane peaks in marine snow samples collected from the bottoms test barrels at the end of the study (FIG. 11) combined with the day 1 TPC data (FIG. 10) indicates that the alkanes had been degraded during the study period.

At each sampling period, a small subsample (i.e., 100-150 mL) was collected for vacuum filtration through a 0.1-μm filter. The filter was then frozen in a vial after addition of RNAlater® (RNAlater is a registered trademark of Ambion Inc., Austin, TX, USA) for 16S rRNA gene amplicon analyses at the completion of the study. At the end of the study, samples of biofilms present on the sides and on the bottom of each barrel were collected and stored frozen after the addition of RNAlater®.

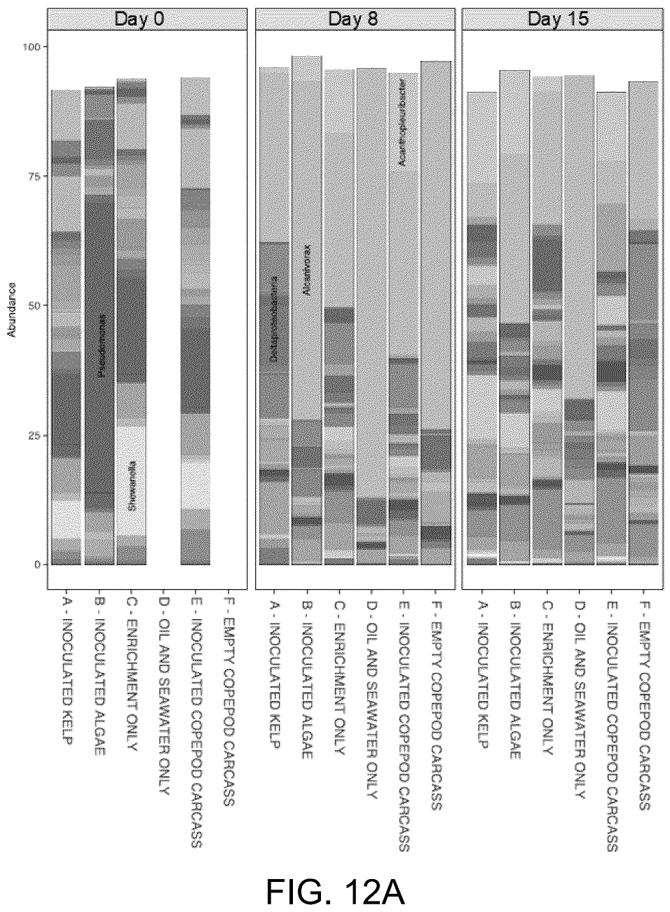

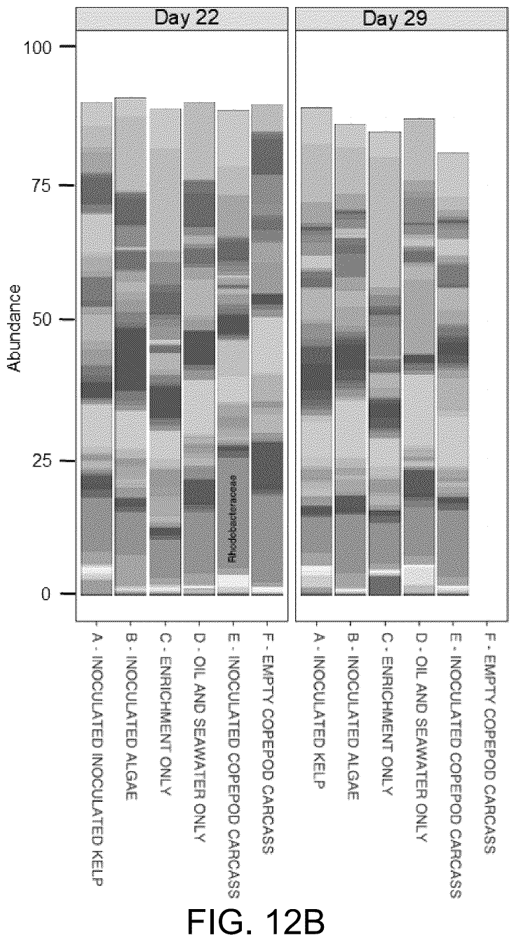

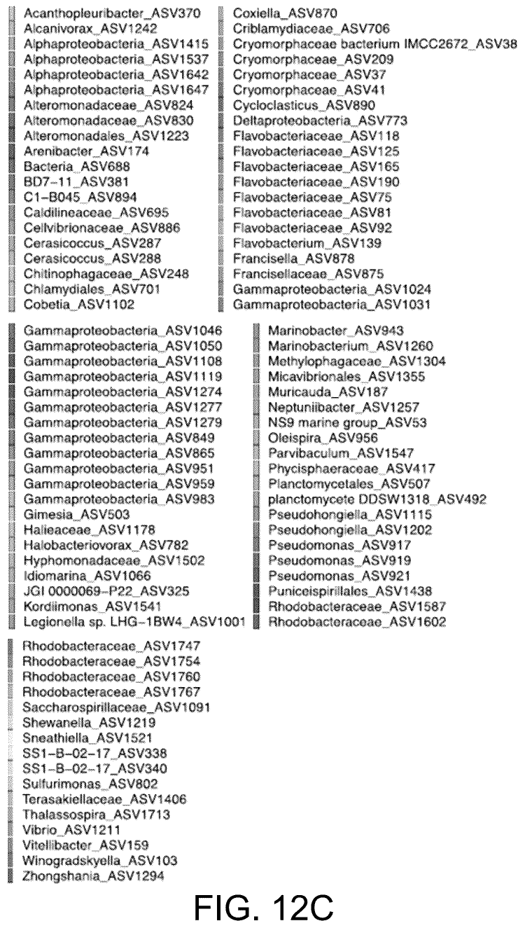

The 16S rRNA gene amplicon data revealed a large shift in community composition when comparing the samples of enrichment and inoculated carriers (day 0) to the barrel samples obtained from day 8 to day 29 (FIGS. 12A, 12B, 12C). Both sample groups contain hydrocarbon degrading organisms, however, different organisms dominate. For instance, the enrichment and inoculated carriers contain high proportions of Pseudomonads, which are only present at low numbers in the barrel samples, while Alcanivorax was present in high numbers in all the barrels, particularly at day 8 (FIGS. 12A, 12B, 12C). This was likely due to the community being enriched on diesel oil, while crude oil was added as the degradation substrate to the barrels. The community was likely therefore not optimally adapted for crude oil degradation. The high numbers of Alcanivorax in all the barrels regardless of enrichment being added, indicates that this organism was present, probably at relatively high levels, in the seawater added to the barrels.

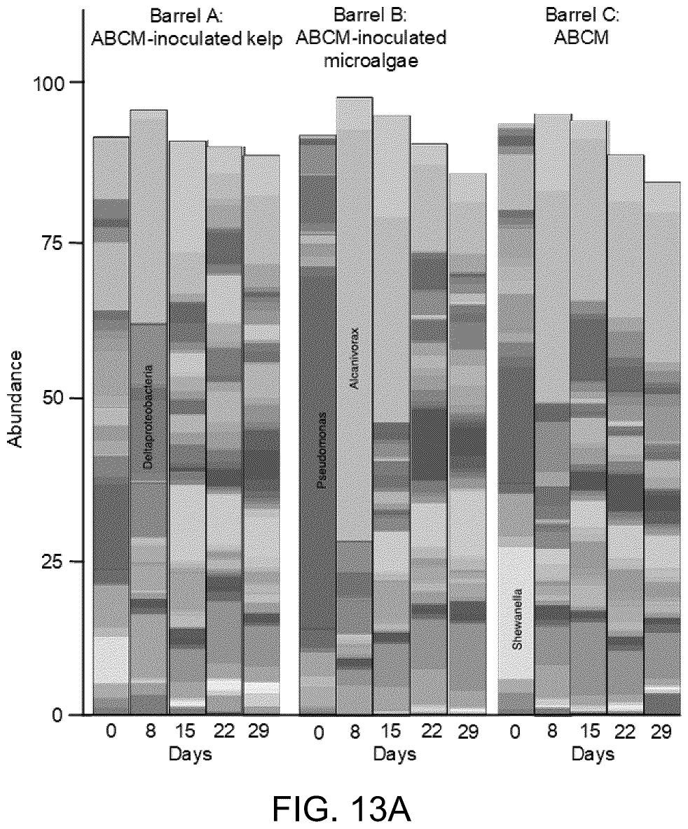

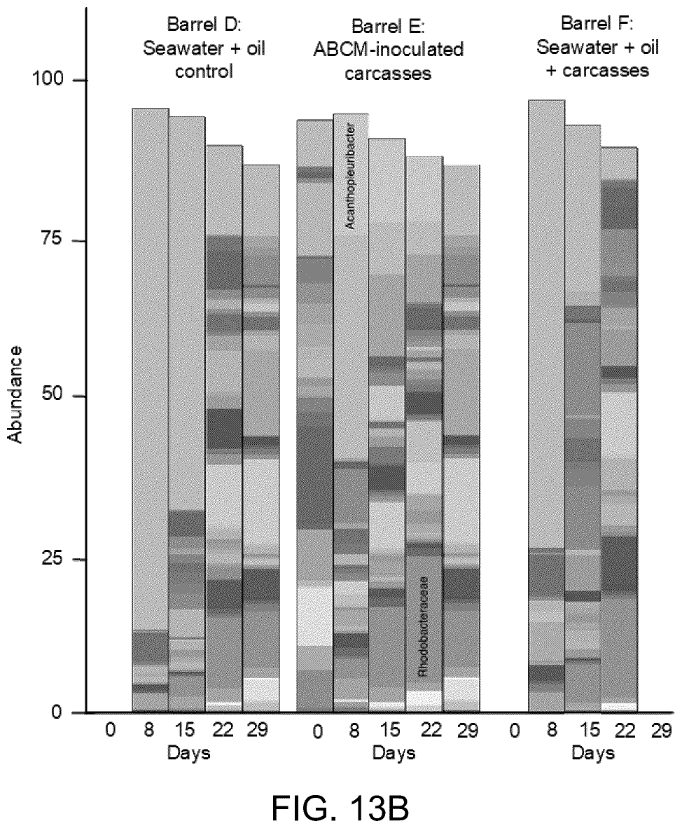

The second major pattern to emerge from the 16S rRNA amplicon data, is a community succession over time, from the very high initial proportions of Alcanivorax at day 8 to a more diverse community (FIGS. 12A, 12B, 12C, 13A, 13B). This process was most rapid in the barrels where the ABCM treatments were added into barrels A, B, C (enriched microbiome), E (FIGS. 12A, 12B, 12C, 13A, 13B), with the highest end-point-diversity at day 29 in barrel E with ABCM copepod carcasses carrier (FIGS. 12A, 12B, 12C, 13B). This strongly suggests that the ABCM contributes significantly to the community in the barrels, even when it has been optimized for a different type of oil. Moreover, the fact that oil first disappeared from barrel E, indicates a positive effect of a more diverse community.

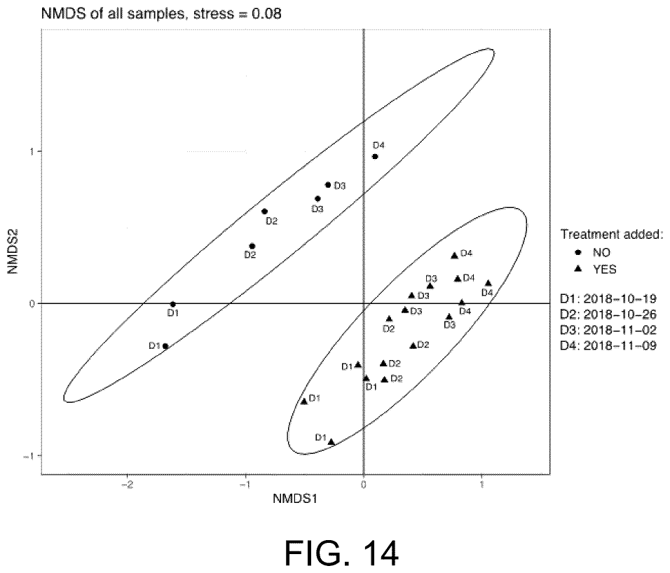

Non-metric dimensional scaling analysis of the 16S rRNA amplicon data shown in FIG. 14, indicates that the development of microbial communities in the control barrels (shown as circles in the upper two quadrants in FIG. 14) was significantly different from the patterns of microbial communities that occurred in the barrels that received enrichment or the ABCM compositions (shown as triangles in the bottom two quadrants and the upper right quadrant in FIG. 14). The numbers in FIG. 14 indicate the sampling day (i.e., D1, D2, D3, D4) while the ellipse denote clustering at a 95% confidence level).

It should be noted that the seawater used in this study was collected from a chronically polluted harbour location and therefore comprised an inherent native microbiome with a variety of acclimated hydrocarbon-degrading microbial species that were capable of degrading the oil slicks added to the positive control barrels D (seawater plus an oil slick only) and F (sea water plus an oil slick plus empty copepod carcasses) (FIGS. 9D, 9E, 13B).

### Example 4

This study assessed the scalability of performance of ABCM compositions to degrade crude oil in larger volumes of marine water collected from the North Sea region of the Atlantic Ocean and having salinity of 30 PSU or greater. Issues considered in this study included determining: (i) the effects of ABCM compositions when applied to simulated oil slicks on the surfaces of Arctic seawater in 200-L barrels in cold temperatures ranging between 4° C. to 10° C., and (ii) determining the effects of larger-volume open systems, gas exchange, evaporation, and water-column mixing on the ABCM performance.

Seawater used for enrichment of a hydrocarbon-degrading microbial community therein, was collected (100 L) from a small commercial harbour in Tromsø, Norway and subsampled into 10 L volumes. The selected commercial harbour has been and remains chronically polluted with petroleum hydrocarbon fuels discharged from commercial vessels. The seawater sample was filtered through a 150-μm mesh screen and then distributed into four individual 5-L blue cap bottles, with approximately 2.5 L of filtered seawater in each bottle.

To enrich the growth of oil-degrading microorganisms in the filtered seawater, 180 mL of ULA crude oil collected from the Greater Ekofisk Area in the North Sea (a common crude oil shipped in this region) was added to each bottle at a ratio of 0.5 mL per litre of seawater (1.25 g crude oil per 2.5 L seawater sample). To ensure a high growth of microorganisms, two enrichments were prepared in parallel. The first enrichment was prepared with Bushnell Haas nutrient broth (VWR International, Mississauga, ON, CA) added at a ratio of 0.5 g per litre of seawater (1.25 g BH broth per 2.5 L seawater sample). The second enrichment was prepared with Felles-Kjøpet (FK) nutrient broth (Felleskjøpet Agri, Tromsø NO) added at a ratio of 0.5 g per litre of seawater (1.25 g FK broth per 2.5 L seawater sample). Additional crude oil and nutrients were added and the headspace air was renewed from each 5-L bottle at 2-day intervals for a 28-day enrichment period. The temperature during the 28-day enrichment period was maintained at 10° C.

### Preparation of ABCM Carriers

Five treatments were assessed in this study as shown in Table 2. A single carrier, copepod carcasses (in ground form, produced at industrial scale as aquarium food), was assessed in two of the treatments (Table 2).

TABLE 2

| Treatment | Carrier | Enrichment | Seawater | Total Volume |
|---|---|---|---|---|
| 1. A: Seawater (control) | — | — | 4.6 L | 4.6 L |
| 2. B: Seawater + oil | — | — | 4.6 L | 4.6 L |
| 3. C: Seawater + oil + enrichment | — | 2 L | 2.6 L | 4.6 L |
| 4. D: Seawater + oil + carrier | 1.5 g | — | 4.6 L | 4.6 L |
| 5. E: Seawater + oil + enrichment + carrier | 1.5 g | 2 L | 2.6 L | 4.6 L |

Each of the five treatment volumes was divided into 4 aliquots with each aliquot dispensed into a 5-L bottle, after which, additional seawater was added to bring the total volume in each bottle up to 4.6 L (Table 2). The 20 bottles were then equidistantly spaced around a plankton wheel and incubated for 24 hr at about 10° C.) under slow rotation (about 3 RPM) of the plankton wheel.

### Preparation, Inoculation, and Monitoring of the Test Barrels

Fresh seawater (2,700 L) was collected from Tromsø channel, Norway at approximately 69°45′16.2″N 19°01′56.6″E, from a seawater intake pipe at the study site. This location is situated some distance from the main shipping port of Tromsø and was used to simulate the effects of a small spill in a non-chronically contaminated area of the channel. 180 L of seawater was transferred into each of the fifteen 200-L test barrels. The seawater was stored in darkness at temperatures ranging from about 4° C. to about 10° C. with 100 L/h aeration until the treatments were added and the study period commenced.

Fifteen 200-L barrels were prepared as illustrated in FIG. 6 and maintained in a cold storage room (4° C. to 10° C.) until the study commenced. Each barrel was provided with a 230V 100 l/h aquarium circulation pump mounted about 25 cm above the bottom of the barrel to provide horizontal circulation of the seawater. A 12V DC groundwater sampling pump equipped with a 10 mm polypropylene tubing for sampling, was mounted about 50 cm above the bottom. An air stone connected to a 4 mm silicone tube, was inserted through the center of the lid to a depth of about 5 cm above the bottom to provide about 100 l/h of air for the purpose of providing oxygen and vertical water circulation throughout the seawater. Sampling tubes and electrical cords connected to electrodes were passed through a 1×4 cm hole at the periphery of the lid to enable the lid to remain fixed-in-place during the duration of the study period to prevent photodegradation and particulate contamination of the barrels. The electrodes were selected to measure (1) water temperature, (ii) oxygen saturation, (iii) eH, (iv) pH, and (v) conductivity.

To each of the fifteen 200-L barrels was added 180 mL of ULA crude oil to simulate an oil slick, followed by 30 g of Felles-Kjopet (FK) nutrient broth after which, the pumps and air stones were turned on to provide mixing of the nutrient broth throughout the 180 L of seawater. Then, the treatments were dispensed into the barrels in triplicates as follows:
Treatment 1 (seawater controls): barrels A1, A2, A3;
Treatment 2 (seawater+oil): barrels B1, B2, B3;
Treatment 3 (seawater+oil+enrichment): barrels C1, C2, C3;
Treatment 4 (seawater+oil+carrier): barrels D1, D2, D3;
Treatment 5 (seawater+oil+enrichment+carrier): E1, E2, E3.

The barrels were sealed with constant supply of oxygen and vertical circulation of the seawater. “ULA crude oil” is considered to be a paraffinic oil with a relatively high content of wax and asphalthenes. The treatment barrels were maintained from about 4 ° C. to about 10° C. for the duration of the study period.

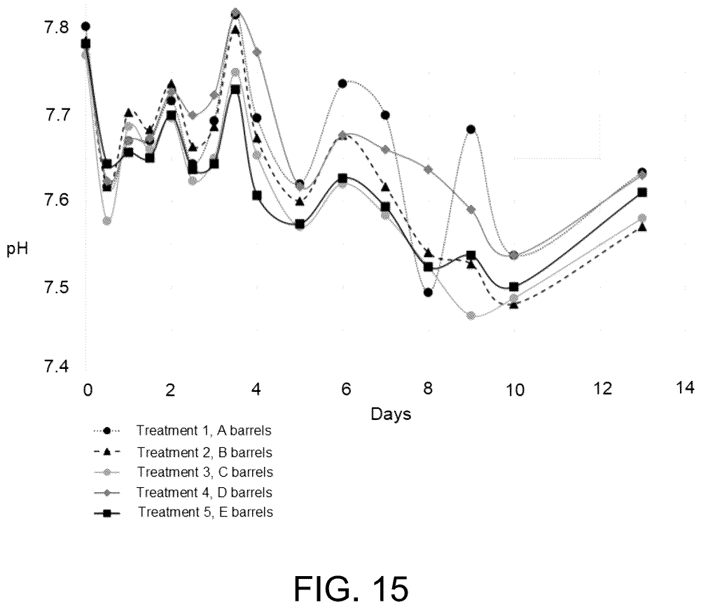

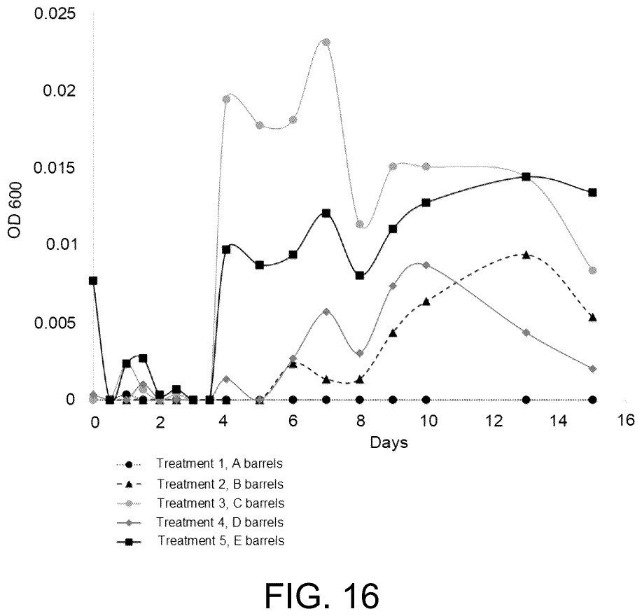

Data collected by the electrodes were recorded: (i) just prior to the addition of the microbial treatments and crude oil slick, (ii) twice daily during the first three days of the study, (iii) then daily (i.e., every 24 h) for 7 days (i.e., through 9 days after the treatments and oil were added to the barrels), and (iv) then once every 7 days for the duration of the 5-wk study period. FIG. 15 shows that none of the treatments had significant effects on seawater pHs during the first 15 days of the study. However, the ABCM treatments in barrels 3 and 5 showed a sharp rise in optical density (OD) measurements, indicative of microbial growth and activity, by the 4^{th} day of the study to levels that remained constant through the 15^{th} day (FIG. 16). The optical density measurements showed some minor evidence of microbial activity commencing after 6 days, in the barrels receiving an oil slick (barrels B) and in the barrels receiving an oil slick and un-inoculated carriers (barrels D) (FIG. 16). No microbial activity was observed in the negative control barrels A (no oil slick or un-inoculated carriers or ABCM) (FIG. 16). It was noted that the oil slicks had disappeared from the tops of barrels that received the ABCM treatments by the 24^{th} day of the study, that is, barrels C and E. However, oil slicks still remained on the water surfaces in barrels A (negative control), B (positive control), and D (received an un-inoculated carrier treatment)

Water samples (500 mL) were collected from each of the barrels from each of the five treatments for analyses of their hydrocarbon contents at daily intervals for the first ten days of the study, then three times weekly during the next three weeks, and then one week later at the end of the 38-day. At that time, the circulation and aeration systems in each barrel were turned off; suspended residue and particulate matter were allowed to finish settling out or sink to the bottom as marine snow, for one to two days. In the barrels that received an oil slick (treatments B, C, D, E), the settled-out particulate matter would be considered to be oil-associated marine snow. The control treatment A that did not receive an oil slick, the settled-out particulate matter would be considered to be a naturally occurring marine snow. Any material on-bottom after pumping out or decanting was separated into two subsamples with one subsample frozen for future hydrocarbon analysis, on the other subsample filtered, fixed with RNAlater®, and then frozen for future DNA isolation/extraction and 16S rRNA gene sequencing.

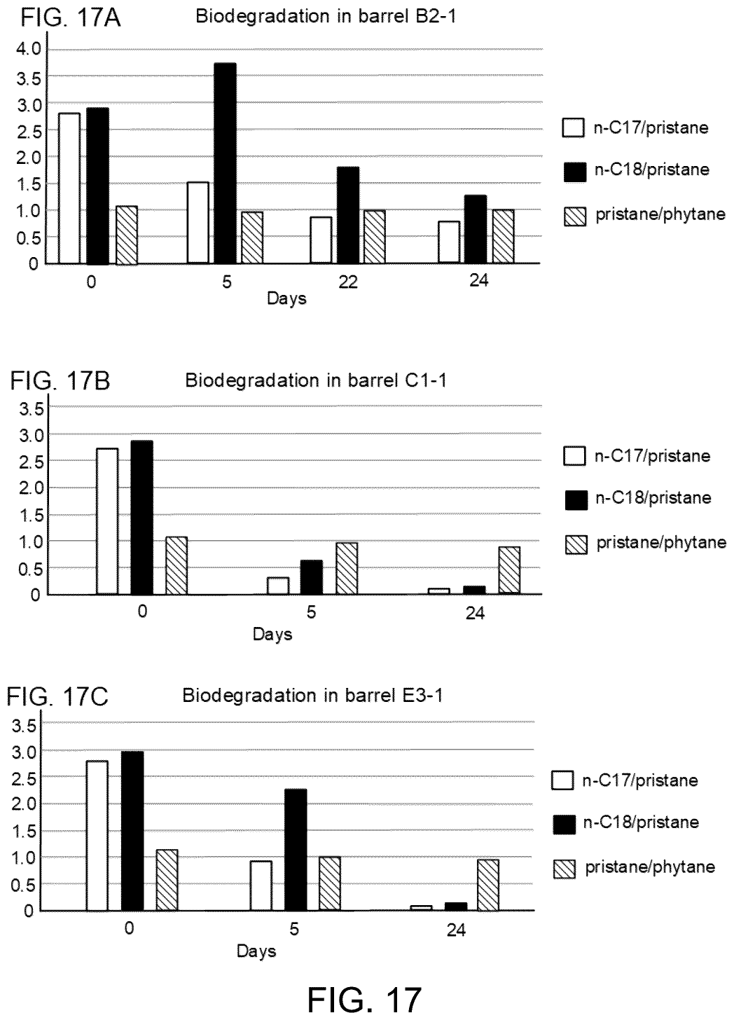

Aliquots of the water samples were analyzed with GC-MS hydrocarbon fingerprinting equipment and methods to determine and characterize biodegradation of the oil slicks in the different treatment barrels. The control reference sample against which the water samples were compared was designated as “K2” and was prepared by adding some of the crude oil used as the slicks, to a seawater sample and then performing the same extraction procedure that was used for the test water samples. The peak height or area of each sample (including the K2 reference sample) was normalized against the peak height of 30ab-hopane in that sample. Example data for changes in reduction of n-C17/pristane and n-18C/phytane ratios compared to the K2 reference sample at time 0, 5 days, 22 days, and 24 days are shown in (i) FIG. 17A for seawater plus oil slick control barrel treatment B2-1, (ii) FIG. 17B for seawater plus oil slick plus ABCM treatment barrel C1-1, and (iii) FIG. 17C for seawater plus oil slick plus ABCM treatment in a carrier barrel E1-1. The data in FIGS. 16B and 16C show that n-C17/pristane and n-C18/phytane ratios were reduced between the day 5 and the day 24 samples. However, the ratios of pristane vs phytane was unchanged, as was expected, because these isoprenoids are resistant to biodegradation.

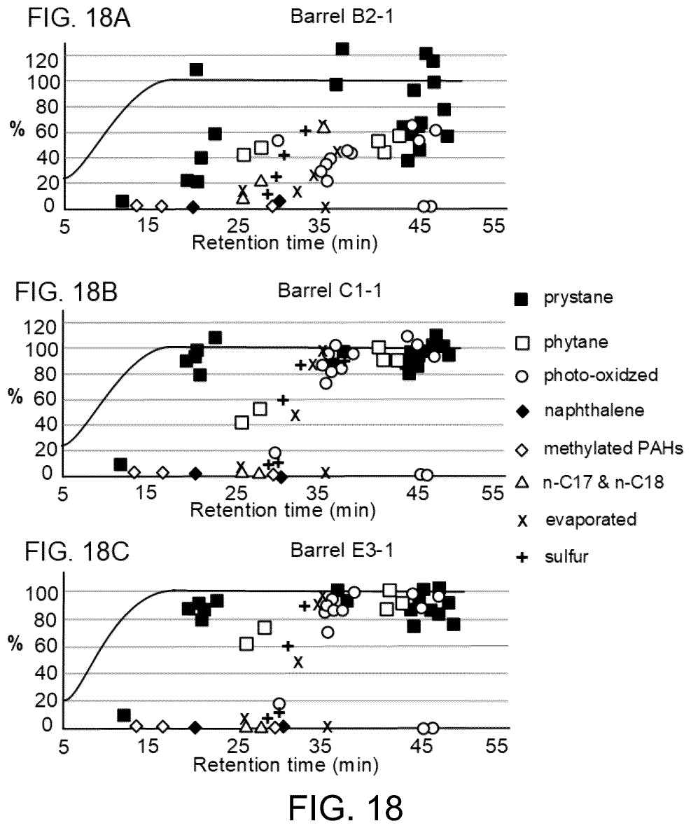

The presence of hydrocarbon breakdown byproducts in the treatment barrels after 28 days was assessed in reference to a “ULA crude oil” sample with GC-MS equipment and methods. The peak height or area of each sample (including the ULA reference sample) was normalized against the peak height of 30ab-hopane in that sample. Each treatment sample was compared with the ULA reference sample, compound by compound and plotted as retention time v. % relative response (FIGS. 18A, 18B, 18C). Retention time (X-axis) reflects the boiling point of each compound. The % relative response (Y-axis) shows intensity of a compound in one sample compared to the same compound in the reference sample. Identical/similar samples will provide results of about 100±20% rel. response. Relative response values less than 100% (or less than 80%) indicates that the concentration of a specific compound in one sample decreased in relative to the reference sample. Similar compounds were given a specific symbol and color indicative of the most likely type of weathering, for example, the blue closed diamonds and open diamonds reference compounds that were lost mainly through dissolution into the water phase; red closed and open circles are indicative of photo-oxidation; green triangles were indicative of biodegradation.

Aliquots of the water samples collected from treatment barrels B2-1 (seawater plus oil slick control), C1-1 (seawater plus oil slick plus ABCM treatment) and E3-1 (seawater plus oil slick plus ABCM in a carrier treatment) after 8 days and after 24 days were analyzed by GC-FID to determine the presence of total hydrocarbons (THC) therein. After 8 days, the levels of THC were rather low in the range of about 25-400 pA. After 24 days, the levels of THC in the two ABDM treatments C1-1 and E3-1 increased dramatically to a range of about 1000-3500 pA whereas the THC levels remained very low in a range of less than 100 pA.

At each sampling period, a small subsample (i.e., 100-150 mL) was collected for vacuum filtration over a 0.1-μm filter. The filter was then frozen in a vial after addition of RNAlater® for 16S rRNA gene amplicon analyses at the completion of the study. At the end of the study, samples of biofilms present on the sides and on the bottom of each barrel were collected and stored frozen after the addition of RNAlater®.

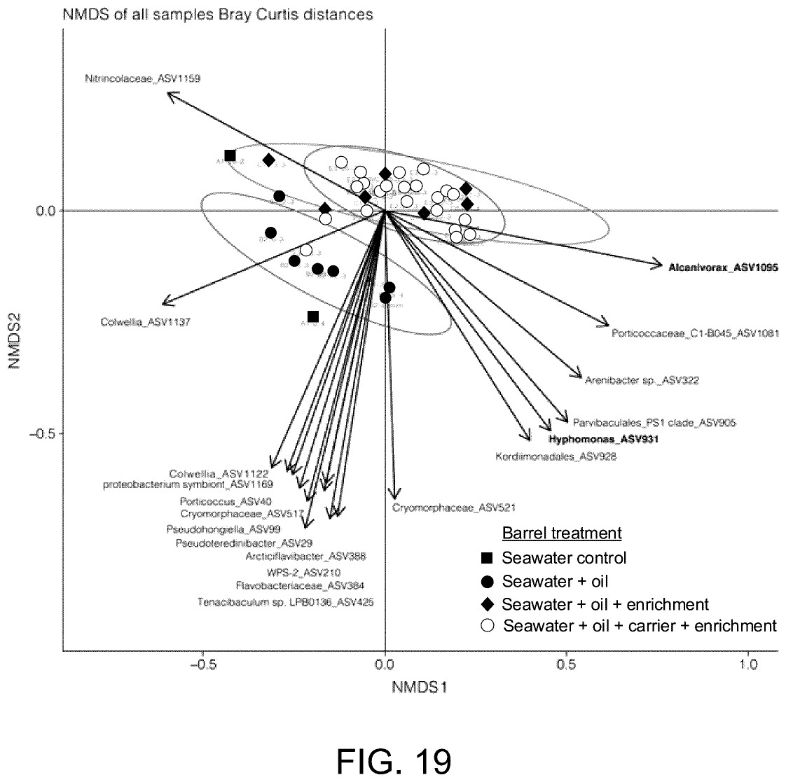

Non-metric dimensional scaling analysis of the 16S rRNA amplicon data shown in FIG. 19, indicates that the development of microbial communities in the control barrels (shown as black squares and black circles in the lower two quadrants in FIG. 19) was significantly different from the patterns of microbial communities that occurred in the barrels that received enrichment or the ABCM compositions (shown as black diamonds and open circles in the upper two quadrants in FIG. 19).

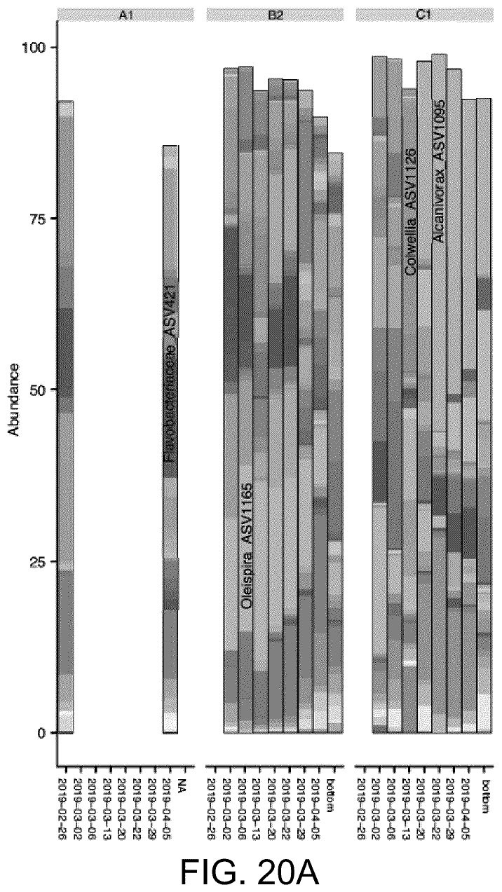

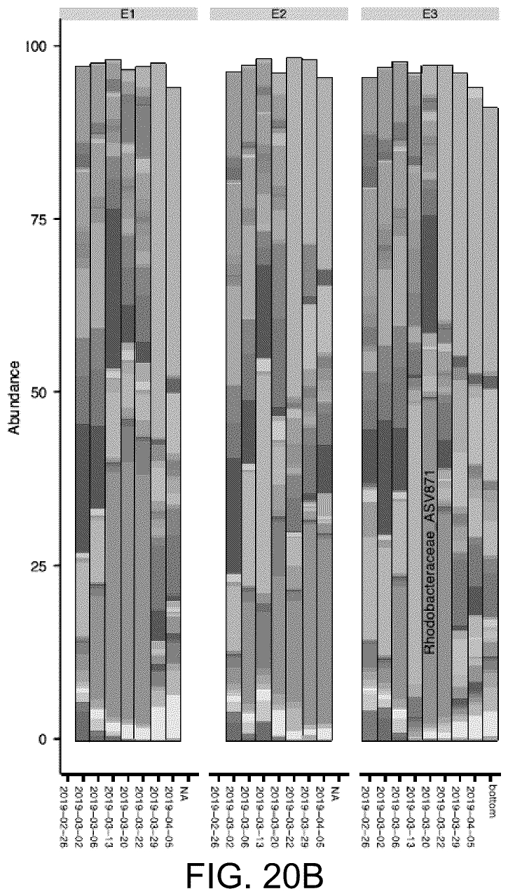

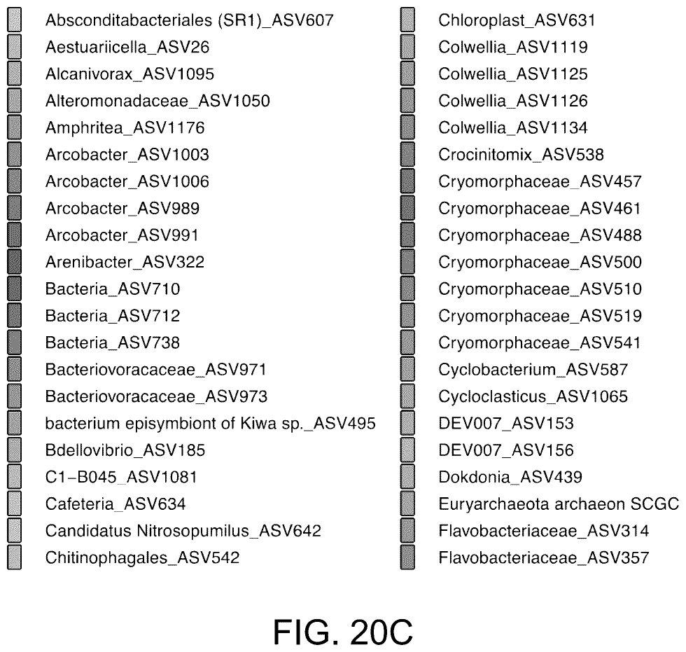

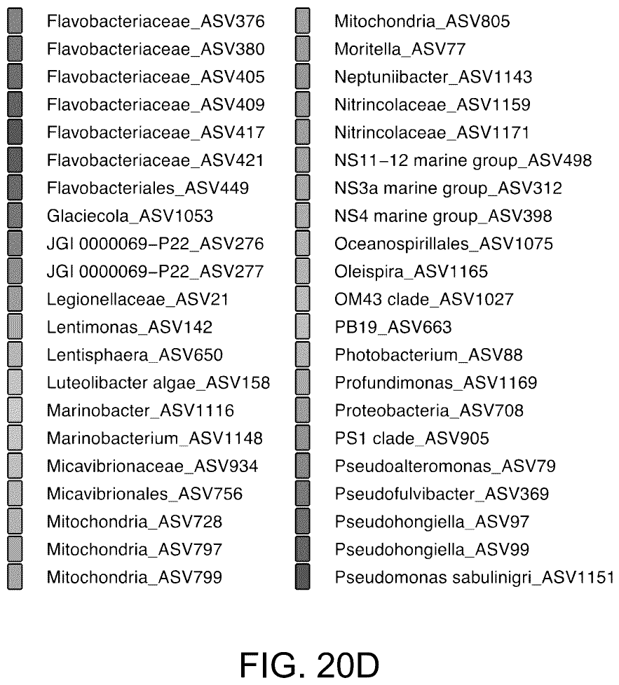

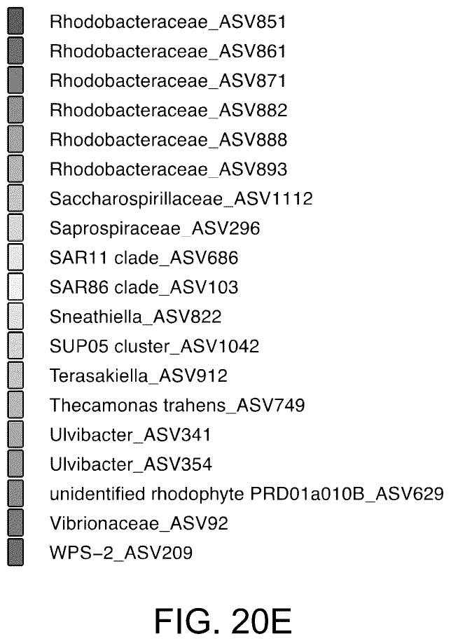

The 16S rRNA gene amplicon data revealed a large shift in community composition during the 38-day study when comparing the water samples from the negative control barrel A1 and the positive control barrel B2, with the water samples from barrel C1 which received a ABCM treatment and from barrels E1, E2, E3 that received ABCM in a carrier treatment (FIGS. 20A, 20B, 20C, 20D, Both sample groups contain hydrocarbon-degrading organisms, however, different microorganisms dominated. These data show that the amplicon sequence variants (ASVs) are highly specific for barrels where enrichment was added, particularly Alcanivorax which becomes dominant in the Cl-barrel 20/3-2019 and in the E-barrels 22/3-2019 — 29/3-2019 (FIGS. 20A, 20B, 20C). Alcanivorax was not present at high levels in the enrichment samples and therefore, was probably recruited from the ‘rare’ biosphere in the enrichments or the water added to the barrels. Alcanivorax never reached high levels in the oil-only barrel B-2 (FIG. 20A). If this organism was introduced by the seawater or by the oil, it could be due to syntrophic effects of the microbial community supplied by the enrichments. It could also be due to lack of micronutrients in the FK-nutrients added to the barrels. Such micronutrients would have been supplied with the enrichments to the C and E barrel. Finally, the differences in succession patterns could be due to a combination of biotic and abiotic effects. The enrichments contain high levels of Oleispira (Oil+FK-nutrients) and several Colwellia species (both FK and BH nutrients).

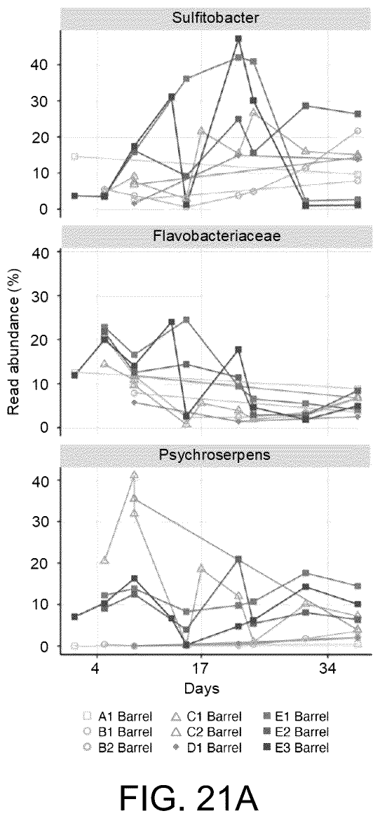

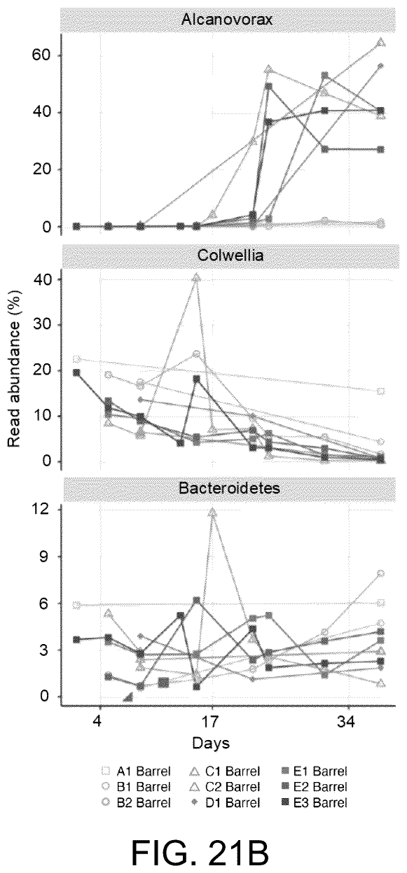

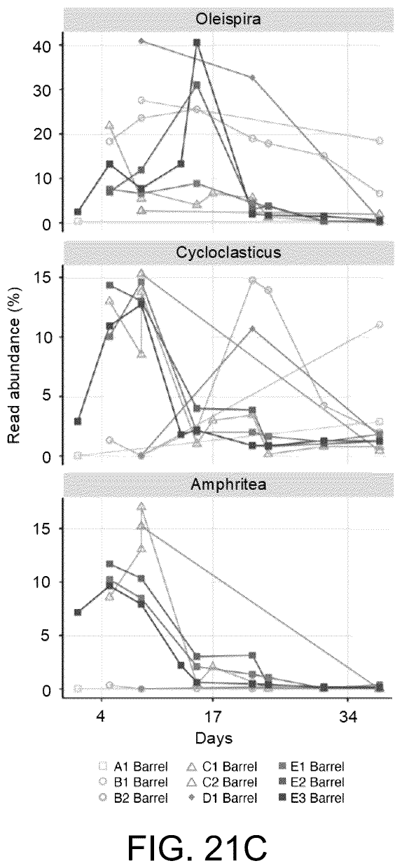

The NMDS in FIG. 19 have taxa that are significantly correlated with the samples added. Again, this illustrates that Alcanivorax is significantly correlated with the C and E later time points. It also shows that a Hyphomonas is correlated with the later time points (B, C and E). Interestingly, organisms from this biofilm associated lineage was also observed associated with addition of enrichment in the Copenhagen barrels (Example 3). These microorganisms were not supplied with the enrichments and were found in the ‘bottom’ samples of B, C and E samples, and it therefore likely part of the ‘natural’ marine snow that formed in the Tromsø barrels. FIGS. 21A, 21B, 21C show time-series plots of the top nine taxa observed in the barrels E1, E2, E3 that received the seawater+oil+enrichment+carrier treatment. These data show that the hydrocarbon-degrading taxa Oleispira (FIG. 21C), Cycloclasticus (FIG. 21C), and Sulfitobacter (FIG. 21A) reach high abundance before the sharp increase in Alcanivorax (FIG. 21B). Interestingly, Sulfitobacter (FIG. 21A) and Colwellia (FIG. 21B) which were supplied from the enrichment, have also been reported to be part of the rapid responder communities to crude oil spills in earlier mesocosms studies under similar conditions. The presence of these taxa, or the functions they carry out, might be important for the establishment of the later community that is dominated by Alcanivorax.

It is to be noted that the microbial communities in seawater used for multiple enrichments for producing the ABCM treatments applied in barrels C and E came from a chronically polluted harbour location next to a re-fueling dock at Skattora small-vessel harbour in Tromsø, were different from microbial communities in the seawater used for testing in barrels A, B, C, D, E. The data in FIGS. 20A-20E show that the microbial community in seawater from Skattora harbour at the end of enriching on diesel fuel and ULA crude oil contained known hydrocarbon-degraders at high % abundance. These data (FIGS. 20A-20E) reveals that water-phase communities in barrels with ABCM treatment (barrels C and E) and barrels without treatment (barrels A, B, D) were different at the end of the 38-day study, and that the differences were detectable with 14 days and further unfolded as the testing progressed.

Microbial succession began in these barrels almost immediately, probably during the first week of testing, based on the sequencing results shown in FIGS. 20A-20E, and succession further continued through to the end of the 38-day study. The pattern is apparent in the % abundance time-series histograms in all of the replicates that were sequenced from the barrels involved. The microbial succession patterns observed were reproducible and consistent over the time-series in cold seawater (from about 4° C. to about 10° C.). These data further confirm that these microbial succession patterns were induced or prompted after final enrichment with ABCMs only (barrels C), or by ABCM-inoculated carriers (barrels E), were added to seawater whereonto crude oil slicks had been added to. Nothing comparable was observed in the control barrels A, B, D.

### Example 5

This study assessed the production of ABCM (active biological countermeasures) compositions and their degradation of selected crude oil components in Pacific Ocean seawater.

The seawater used in this study was collected from four sampling sites alongside the tanker loading dock at the Westridge Marine Terminal on the south shore of Burrard Inlet at approximately 49°17′27.2″N 122°56′59.8″W in Burnaby BC, in the month of August. The Westridge Marine Terminal was constructed in the early 1950s as the terminal end of the Trans Mountain Pipeline that starts in Edmonton Alberta, and has been used continuously since the mid 1950s to load ocean vessels with crude oil or semi-crude oil or refined petroleum products across the Pacific Ocean to west coast USA and Asia. The Trans Mountain Pipeline is the only pipeline in North America that carries refined product and crude oil in batches. An example of an unrefined heavy crude oil commonly transported to the Westridge Marine terminal is diluted bitumen. A significant portion of the Trans Mountain Pipeline batches is Alberta convention crude oil classified as an unrefined light crude oil. Other high-volume batch-conveyed products include Bunker C fuel, a residual thick and viscous fuel oil, and summer diesel fuel. Consequently, the waters adjacent to the tanker docks have become chronically polluted with the petroleum hydrocarbons comprising these products.

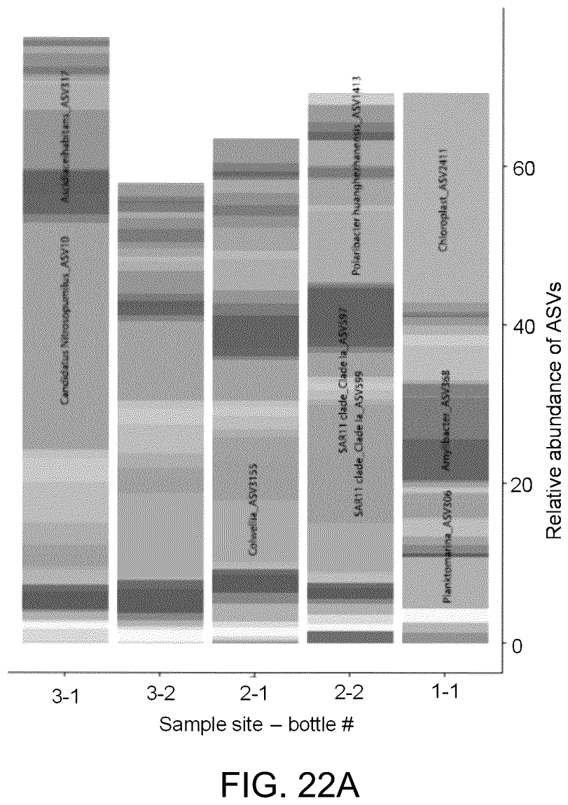

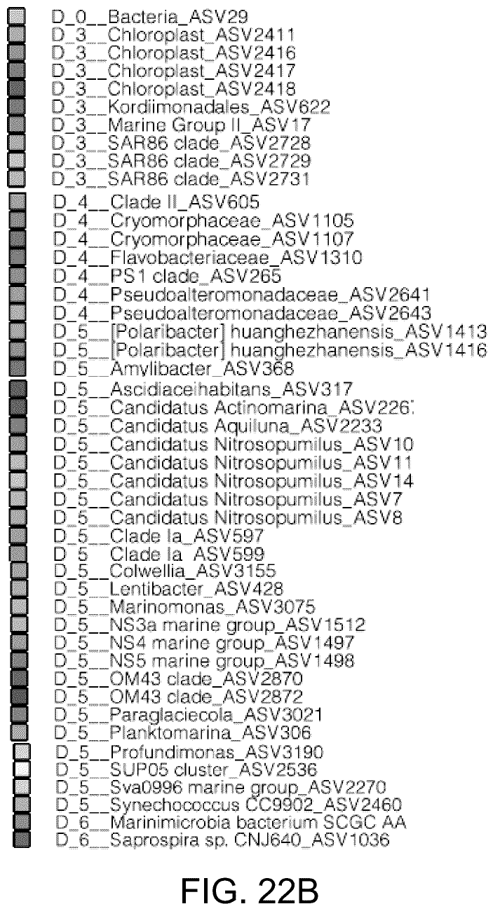

Two 4 L bottles of seawater were collected from each of the four sampling sites adjacent for a total of eight bottles with a total of 32 L. A subsample was collected from each 4 L bottle and vacuum filtered through a 0.22 μm filter. The filters were then frozen in vials after addition of RNAlater® for 16S rRNA gene amplicon and analysed at the completion of the Example 5 studies. The data in FIGS. 22A, 22B shows the relative abundance of amplicon sequence variants (ASVs) from five of the sixteen 2 L seawater samples collected from the Westridge Marine Terminal.

### Enrichment of Naturally Occurring Oil-Degrading Microbiomes

500 mL samples of the dockside seawater collected from the Westridge Marine Terminal were dispensed into eight treatment bottles and numbered as listed below. A ninth 500 mL sample was filtered through a 22 μm filter and designated as a negative control (treatment 1.1). The treatment bottles were prepared as follows:
Treatment 1.1 (Control 1): 500 mL filtered seawater+0.25 mL diesel fuel+0.25 g Bushnell Haas (BH) minerals (water was filtered through a 0.22 μm membrane filter).
Treatment 1.2 (Control 2): 500 mL filtered seawater+0.25 mL diesel fuel+50 mL of a 10×BH nutrient solution (water was filtered through a 0.22 μm membrane filter; the BH nutrient solution was sterilized by autoclaving).
Treatment 2: 500 mL unfiltered seawater+0.25 mL Alberta conventional crude oil+0.25 g BH minerals.
Treatment 3: 500 mL unfiltered seawater+0.25 mL diesel fuel+0.25 g BH minerals.
Treatment 4: 500 mL unfiltered seawater+0.25 mL bunker C fuel+0.25 g BH minerals.
Treatment 5.1: 500 mL unfiltered seawater+0.25 mL diluted bitumen+0.25 g BH minerals.
Treatment 5.2: 200 mL unfiltered seawater+0.1 mL diluted bitumen+0.25 g BH minerals in unfiltered seawater.
Treatment 5.3: 200 mL unfiltered seawater+0.1 mL diluted bitumen+5 g BH minerals in unfiltered seawater.
Treatment 5.4: 200 mL unfiltered seawater+0.1 mL diluted bitumen+0.1 g BH minerals.

The treatment bottles 1-5.1 were stirred with magnetic stirrers while bottles 5.2, 5.3, 5.4 were placed onto a shaker table and incubated with gentle shaking in the dark at ambient temperatures for 60 days. Subsamples were collected from each treatment bottle after 4 days for optical density measurements at 600 nm, and then weekly thereafter for the duration of the 60-day study period. After three weeks after the study commenced, additional BH nutrient solution was added to each treatment bottle.

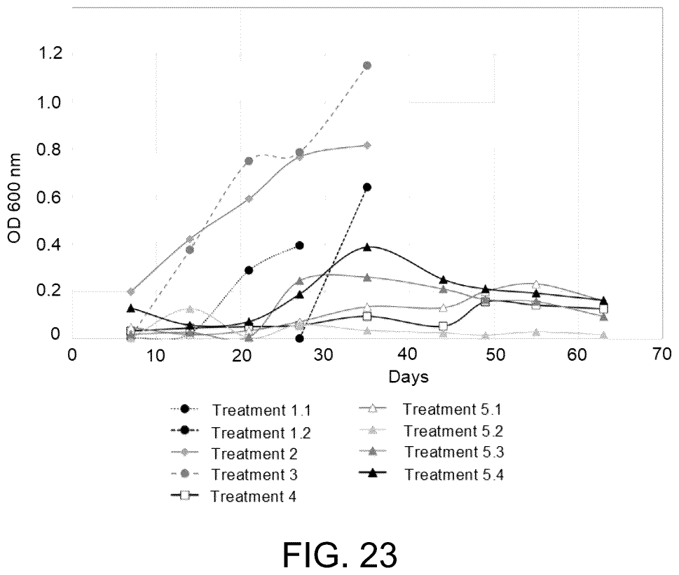

The changes in optical densities that occurred in the nine treatment bottles over the 60-day period are shown in FIG. 23. It was noted during the third week of the study that microbial growth was occurring in the negative control treatment 1.1. Accordingly, a second control treatment 1.2 with 500 mL of filtered seawater to which was added 0.25 mL diesel fuel and 0.25 mL of sterilized BH nutrient solution, and replaced the control treatment 1.1 on the shaker table. However, significant microbial growth occurred in the second negative control treatment 1.2 within 1 week (FIG. 23) suggesting that perhaps the diesel fuel used in this study contained a microbial population.

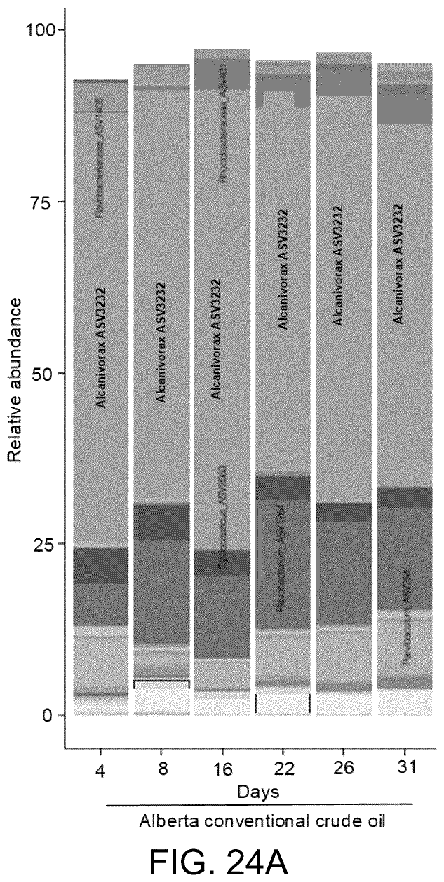

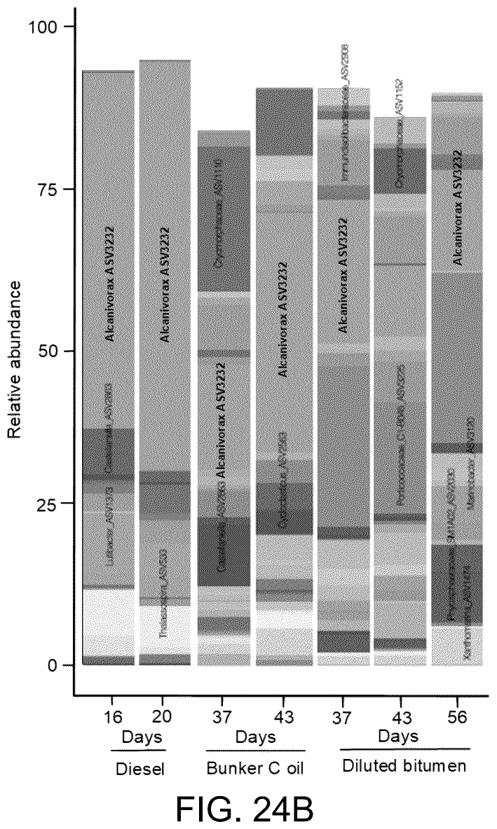

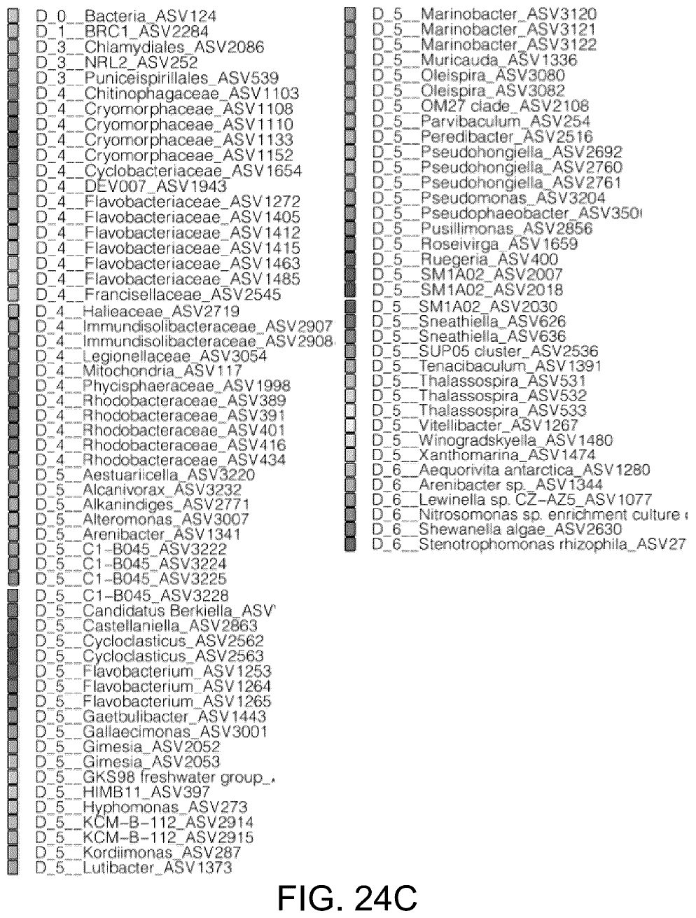

The greatest increase in optical densities during the first five weeks of the study occurred in treatment 2 (supplemented with Alberta conventional crude oil), and in treatment 3 (supplemented with diesel fuel) (FIG. 24). Although some microbial growth was observed in the bottle supplemented with bunker C oil (treatment 4) and in the bottles supplemented with diluted bitumen (treatments 5.1-5.4), the continuing development of microbial populations (as determined by OD measurements) in these bottles at the end of the 60-day period were small in comparison to the microbial activities in treatments 2 and 3 after 4 weeks (FIG. 23). This was partly due to the development of biofilm clumps associated with oil droplets.

### 16S rRNA Gene Amplicon Analyses of Microbiome Populations in the Enrichment ABCM Cultures

At each sampling period, a small subsample (i.e., 2-4 mL) was collected from each bottle and centrifuged at 13,000 rpm. The pellets were transferred into vials and after the addition RNAlater®, were stored at −80° C. for 16S rRNA gene amplicon analyses at the completion of the study. At the end of the study, samples of biofilms present on the sides and on the bottom of each barrel were collected and stored frozen after the addition of RNAlater®.

DNA was isolated from each of the stored samples using the PowerLyse Power Soil kit from Qiagen (Toronto, CA) following the instructions in the kit's manual. DNA concentrations in the samples were determined with a Qubit fluorometer (Thermo Fisher Scientific). The DNA samples were sent to Microbiome Insights (Vancouver, BC, CA) for 16S rRNA amplicon sequencing and metagenome sequencing. Primers used for 16S rRNA amplification were (i) the 515F primer “GTGCCAGCMGCCGCGGTAA”, and (ii) the 806R primer “GGACTACHVGGGTWTCTAAT”. 16S rRNA amplicon data were processed to amplicon sequence variants using the QIIME2 platform (Bolyen et al., 2019,. Reproducible, interactive, scalable and extensible microbiome data science using QIIME 2. Nature Biotechnology 37:852-857) using DADA2-plugin (Callahan et al., 2016, High-resolution sample inference from Illumina amplicon data. Nature Methods 13:581-583) and visualized using the phyloseq in R package for microbiome census data (McMurdie and Holmes, 2013, phyloseq: An R Package for Reproducible Interactive Analysis and Graphics of Microbiome Census Data. PLoS ONE 8:e61217).

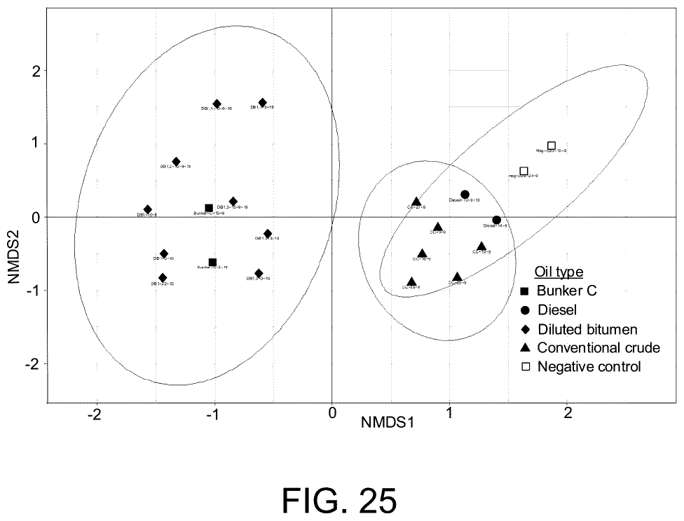

The 16S rRNA gene amplicon data generated from the samples collected during enrichment of the Burrard Inlet seawater microbiome populations (FIGS. 24A, 24B) with Alberta conventional crude oil revealed a large shift in community composition within the first 4 days during which time Alcanivorax ASV3232 emerged as the dominant microbial species in the microbiome accompanied by Cycloclasticus sp., Flavobacterium sp., and Parvibaculum sp. (FIGS. 24A, 24B). The dominance of Alcanivorax ASV3232, Cycloclasticus sp., Flavobacterium sp., and Parvibaculum sp. in the Alberta conventional crude oil enrichments continued for the duration of the 31-day study period (FIGS. 24A, 24B). Alcanivorax ASV3232 also was established as a dominant microbial species in (i) the ABCM microbiome that was established in 16-20 days with the diesel oil enrichment treatment, (ii) the ABCM microbiome that was established in 37-43 days with the bunker C oil enrichment treatment, and (iii) the ABCM microbiome that was established in 37-56 days with the diluted bitumen enrichment treatment (FIGS. 25B, 25C).

Non-metric dimensional scaling analysis of the 16S rRNA amplicon data shown in FIG. 25, indicates that the development of microbial communities in the enrichment with Alberta conventional crude oil was similar to the microbial communities that developed with the diesel oil enrichment (shown within the upper and lower ellipses, respectively, in the two quadrants on the right side of FIG. 25, but were significantly different from the microbial communities that developed with the bunker C oil and diluted bitumen enrichments (shown within the ellipse in the two quadrants on the left side of FIG. 25).

### Degradation of Selected Unrefined Crude Oil Products by Selected Enriched Oil-Degrading Microbiomes

In the first degradation study, 100 mL of enriched microbiome that was produced in treatment 2 (supplemented with Alberta conventional crude oil and having an OD₆₀₀ of 0.658) after four weeks in the enrichment study disclosed above, was combined with 0.07 g bull kelp powder (obtained from BC Kelp, Prince Rupert, BC, Canada) and 400 mL filtered seawater, and then incubated on a plankton wheel at 2-3 rpm at ambient temperatures for about 18 h during which time, enriched microbiome-carrier aggregates were produced that were similar to those produced in Example 1. The enriched microbiome-carrier aggregates were concentrated by passing the culture through a 0.4 μm filter, and using the retentate as the ABCM treatments

The first degradation study comprised two treatments assessing ABCM degradation of Alberta conventional crude oil wherein treatment 1 assessed ABCM performance in unfiltered seawater collected from alongside the tanker loading dock at the Westridge Marine Terminal, while treatment 2 assessed ABCM performance in a filtered portion of the collected seawater. Treatment 1 consisted of (i) a 0.9 L volume of unfiltered seawater, plus (ii) 100 μL of a 0.25 mg/mL BH nutrient solution, plus (iii) 0.5 mL of Alberta conventional crude oil, plus (iv) 200 mL of the concentrated ABCM treatment. Treatment 2 consisted of (i) a 0.9 L volume of filtered seawater, plus (ii) 100 μL of a 0.25 mg/mL BH nutrient solution, plus (iii) 0.5 mL of Alberta conventional crude oil, plus (iv) 200 mL of the concentrated ABCM treatment. The treatments were added into 2 L bottles and incubated for in the dark at ambient temperature for about 4 weeks on a shaker table at about 2-3 rpm. Subsamples were collected from the bottles at regular intervals and the optical densities of the subsampled treatments were determined at 660 nm. The collected data are shown in Table 3. The data show that microbial populations proliferated throughout the 25-day study in filtered seawater that received an Alberta conventional crude oil slick plus the ABCM treatment. However, it appears that the microbiome population collapsed between day 18 and day 25 in treatment 1 with unfiltered water, perhaps because the nutrient supply was exhausted.

TABLE 3
Optical density measurements (600 nm) pertaining
to ABCM treatment degradation of Alberta conventional
crude oil in filtered and unfiltered seawater

| | Days after ABCM treatment | | | | |
|---|---|---|---|---|---|
| Treatment | 4 | 7 | 14 | 18 | 25 |
| 1. Unfiltered sea water + | 0.282 | 0.254 | 0.298 | 0.300 | 0.032 |
| ABCM + ACC* | | | | | |
| 2. Filtered sea water + | 0.217 | 0.324 | 0.381 | 0.500 | 0.443 |
| ABCM + ACC* | | | | | |
| 3. Negative control | 0.224 | 0.394 | 0.466 | 0.462 | 0.113 |

ACC—Alberta conventional crude oil

In the second degradation study, 50 mL of enriched microbiome produced in treatment 5.1 (supplemented with diluted bitumen and having an OD₆₀₀ of 0.160) after the 60-days in the enrichment study disclosed above, was combined with 0.08 g bull kelp powder and 250 mL filtered seawater, then incubated on a plankton wheel at about 2-3 rpm at ambient temperatures for about 18 h during which time, enriched microbiome-carrier aggregates were produced that were similar in appearance to those produced in Example 1. The enriched microbiome-carrier aggregates were concentrated by passing the culture through a 5 μm filter, and using the retentate as the ABCM treatments.

The second degradation study comprised two ABCM treatments assessing ABCM degradation of diluted bitumen wherein treatment 1 assessed ABCM performance in unfiltered seawater collected from alongside the tanker loading dock at the Westridge Marine Terminal, while treatment 2 assessed ABCM performance in a filtered portion of the collected seawater.

Treatment 1 consisted of (i) a 0.9 L volume of unfiltered seawater, (ii) 100 mL of a 0.25 mg/mL BH nutrient solution, (iii) 0.5 mL of diluted bitumen, plus (iv) 10mL of the concentrated ABCM treatment.

Treatment 2 consisted of (i) a 0.9 L volume of filtered seawater, plus (ii) 100 μL of a 0.25 mg/mL BH nutrient solution, plus (iii) 0.5 mL of diluted bitumen, plus (iv) 10mL of the concentrated ABCM treatment.

Treatment 3 was a control treatment consisting only unfiltered seawater (0.9 L).

Treatment 4 was a first reference treatment consisting of 0.9 L of unfiltered seawater plus 10 mL of the concentrated ABCM treatment.

Treatment 5 was a second reference treatment consisting of 0.9 L of unfiltered seawater plus 0.5 mL of diluted bitumen.

The treatments were added into 2L bottles and incubated for in the dark at ambient temperature for about 5 weeks on a shaker table 2-3 rpm. Subsamples were collected from the bottles at regular intervals and the optical densities of the subsampled treatments were determined at 660 nm. The collected data are shown in Table 4. The data show that low levels microbial populations developed and were maintained throughout the 33-day study in the filtered and unfiltered seawater that received diluted bitumen oil slick plus the ABCM treatment. It was observed that most of the microbial community development in the treatment bottles that received diluted bitumen, occurred as biofilms associated with the bitumen resulting in dispersion of the bitumen into the water column. This type of microbial community development was not observed in the control treatment water bottles.

TABLE 4
Optical density measurements (600 nm) pertaining to ABCM treatment
degradation of diluted bitumen in filtered and unfiltered seawater

| | Days after ABCM treatment | | | | |
|---|---|---|---|---|---|
| Treatment | 4 | 7 | 14 | 18 | 25 |
| 1. Unfiltered seawater + | 0.027 | 0.033 | 0.050 | 0.031 | 0.035 |
| ABCM + DB* | | | | | |
| 2. Filtered seawater + | 0.023 | 0.074 | 0.103 | 0.039 | 0.023 |
| ABCM + DB | | | | | |
| 3. Unfiltered seawater | ND** | ND | ND | ND | ND |
| 4. Unfiltered seawater + ABCM | 0 | NM | ND | ND | ND |
| 5. Unfiltered seawater + DB* | NA*** | ND | ND | ND | ND |

DB—diluted bitumen
ND—not detected
NA—not applicable

### Example 6

### Metagenome sequencing

Based on results from the 16S rRNA analyses in Examples 4 and 5, ten samples were selected for metagenome sequencing. Samples 1 to 3 are time-series samples from ABCM treatment 5 in Example 4 with seawater collected from the Tromsø channel, Norway, sample 4 was a negative control treatment from Example 4, sample 5 was a positive control treatment from Example 4, and samples 6 and 7 were collected on the same date from ABCM treatment 5 in Example 4. Samples 8 to 10 were selected from the treatments in Example 5 with seawater collected from coastal British Columbia, Canada.
Sample 1: sample ID “E3-6-3” from treatment 5 in Example 4 (Table 2) wherein the carbon source was Ula crude oil (treatment 5.1 was 2.6 L unfiltered seawater+180 mL Ula crude oil+2 L Ula-crude-oil-enriched microbiome aggregates+1.5 g ground copepod carcasses);
Sample 2: sample ID “E3-22-3” from treatment 5 in Example 4 (Table 2);
Sample 3: sample ID “E 5/4” from treatment 5 in Example 4 (Table 2);
Sample 4: sample ID “B2-22-3” from treatment 2 in Example 4 (Table 2) that was a control containing only 2.6 L unfiltered seawater+180 mL Uka crude oil;
Sample 5: sample ID “C1-22-3” from treatment 3 in Example 4 (Table 2) containing 2.6 L unfiltered seawater+180 mL Uka crude oil+2 L Ula-crude-oil-enriched microbiome;
Sample 6: sample ID “E1-22-3” from treatment 5 in Example 4 (Table 2);
Sample 7: sample ID “E2-22-3” from treatment 5 in Example 4 (Table 2);
Sample 8: sample ID “DB1-22-10” from treatment 5.1 in the Example 5 enrichment study wherein the carbon source was diluted bitumen (treatment 5.1 was 500 mL unfiltered seawater+0.25 mL diluted bitumen+0.25 g BH minerals);
Sample 9: sample ID “CC-20-9” from treatment 1 in the first degradation study (Example 5) wherein the carbon source was Alberta conventional crude oil (treatment 1 was unfiltered seawater+a microbiome sample from treatment 5.1 in the enrichment study+Alberta crude oil);
Sample 10: sample ID “UF-bottom-22-10” from treatment 1 in the second degradation study (Example 5) wherein the carbon source was diluted bitumen (treatment 1 was unfiltered seawater+a microbiome sample from treatment 5.1 in the enrichment study+diluted bitumen).

Quality control assessment of the metagenome reads (Table 5), show that 8.4 million to 12 million reads for each of the samples were of high quality (over 96%).

TABLE 5

| | | Number of | Total pairs | Total pairs |
|---|---|---|---|---|
| Sample No. | Sample ID | pairs analyzed | passed | passed (%) |
| 1 (Example 4) | E3-6-3 | 13,297,358 | 12,823,036 | 96.4 |
| 2 (Example 4) | E3-22-3 | 13,301,646 | 12,855,316 | 96.6 |
| 3 (Example 4) | E3 5/4 | 12,605,123 | 12,174,104 | 96.6 |
| 4 (Example 4) | B2-22-3 | 13,320,524 | 12,922,386 | 97.0 |
| 5 (Example 4) | C1-22-3 | 11,938,027 | 11,531,600 | 96.6 |
| 6 (Example 4) | E1-22-3 | 12,77,3603 | 12,350,809 | 96.7 |
| 7 (Example 4) | E2-22-3 | 11,456,164 | 11,063,863 | 96.6 |
| 8 (Example 5) | DB1-22-10 | 12,493,819 | 12,027,247 | 96.3 |
| 9 (Example 5) | CC-20-9 | 8,772,030 | 8,465,434 | 96.5 |
| 10 (Example 5) | UF-bottom-22-10 | 9,251,002 | 8,938,244 | 96.6 |

All samples were assembled separately and as co-assemblies using metaspades and the Anvio snakemake workflow (http://merenlab.org/2018/07/09/anvio-snakemake-workflows/). The co-assembly statistics for samples 1 to 7 (north Atlantic, Norway) are shown in Table 6 while the co-assembly statistics for samples 8 to 10 (north Pacific. Canada) are shown in Table 7.

The assemblies were large but mostly made up of short contiguous sequences (contigs). The focus of the analysis was on contigs greater than 1000 bp. Very few read contribute to the numerous small contigs. When the mapping reads from Tromsø (North Atlantic) were correlated back to the constructed genome bins (metagenome-assembled genomes, also referred to as “MAGs”), 84% to 95% of the reads mapped back to the contigs >2,500 bp used in binning (Table 8). When the mapping reads from British Columbia (North Pacific) were correlated back to the constructed MAGs, 92% to 95% of the reads mapped back to the contigs >2,500 bp used in binning (Table 9).

TABLE 6

| Contigs_db | Tromsø, North Atlantic |
|---|---|
| Total Length | 1,017,870,697 |
| Num Contigs | 2,381,646 |
| Num Contigs > 1 kb | 86,173 |
| Num Contigs > 5 kb | 9,884 |
| Num Contigs > 10 kb | 4,913 |
| Num Contigs > 20 kb | 2,095 |
| Num Contigs > 50 kb | 518 |
| Num Contigs > 100 kb | 139 |
| Longest Contig | 685,887 |
| Shortest Contig | 56 |
| Num Genes (prodigal) | 2,236,459 |
| L50* | 436,362 |
| N50** | 422 |

Raw number of HMM Hits

| | |
|---|---|
| Protista_83 | 825 |
| Archaea_76 | 9312 |
| Bacteria_71 | 15179 |
| Ribosomal_RNAs | 60 |
| archaea (Archaea_76) | 5 |
| bacteria (Bacteria_71) | 149 |
| eukarya (Protista_83) | 2 |

Approx. number of genomes

| | |
|---|---|
| eukarya (Protista_83) | 5 |
| archaea (Archaea_76) | 2 |
| bacteria (Bacteria_71) | 149 |

*the L50 count is defined as the smallest number of contigs whose length sum makes up half of genome size
**the N50 is defined as the sequence length of the shortest contig at 50% of the total genome length

TABLE 7

| Contigs Stats | BC, North Pacific |
|---|---|
| Total Length | 314,788,814 |
| Num Contigs | 637,226 |
| Num Contigs > 1 kb | 32,664 |
| Num Contigs > 5 kb | 3,244 |
| Num Contigs > 10 kb | 1,331 |
| Num Contigs > 20 kb | 583 |
| Num Contigs > 50 kb | 248 |
| Num Contigs > 100 kb | 98 |
| Longest Contig | 1,976,102 |
| Shortest Contig | 55 |
| L50 | 87,166 |
| N50 | 535 |

Raw number of HMM Hits

| | |
|---|---|
| Protista_83 | 267 |
| Archaea_76 | 4,126 |
| Ribosomal_RNAs | 27 |
| Bacteria_71 | 7,327 |

Approx. number of genomes

| | |
|---|---|
| eukarya (Protista_83) | 0 |
| archaea (Archaea_76) | 0 |
| bacteria (Bacteria_71) | 76 |

* the L50 count is defined as the smallest number of contigs whose length sum makes up half of genome size
** the N50 is defined as the sequence length of the shortest contig at 50% of the total genome length

TABLE 8
Tromsø (North Atlantic) constructed genome bins

| Sample | | Total no. | Total reads | | No. SVCs |
|---|---|---|---|---|---|
| No. | Sample ID | reads | mapped | % mapped | reported |
| 1 | E3-6-3 | 25,646,072 | 23,206,855 | 90.5 | 428,130 |
| 2 | E3-22-3 | 25,710,632 | 24,223,283, | 94.2 | 121,463 |
| 3 | E3 5/4 | 24,348,208 | 22,220,431 | 91.3 | 180,512 |
| 4 | B2-22-3 | 25,844,772 | 21,894,994 | 84.7 | 330,271 |
| 5 | C1-22-3 | 24,701,618 | 22,003,592 | 95.4 | 180,469 |
| 6 | E1-22-3 | 22,127,726 | 20,669,684 | 87.9 | 161,943 |
| 7 | E2-22-3 | 25,646,072 | 23,206,855 | 93.4 | 197,544 |

TABLE 9
British Columbia (North Pacific)

| Sample | | Total no. | Total pairs | | No. SVCs |
|---|---|---|---|---|---|
| No. | Sample ID | reads | mapped | % mapped | reported |
| 8 | DB1-22-10 | 24,054,494 | 12,027,247 | 92.6 | 279,440 |
| 9 | CC-20-9 | 16.930.868 | 8,465,434 | 95.5 | 27,505 |
| 10 | UF-bottom-22-10 | 17,876,488 | 8,938,244 | 94.8 | 24,238 |

Many environmental microbes are known only from high-throughput sequence data, but the small-subunit rRNA (SSU rRNA) gene, the key to visualization by molecular probes and link to existing literature, is often missing from metagenome-assembled genomes (MAGs). The phyloFlash software suite tackles this gap with rapid, SSU rRNA-centered taxonomic classification, targeted assembly. Starting from a cleaned reference database, phyloFlash profiles the taxonomic diversity and assembles the sorted SSU rRNA reads. The phyloFlash design is domain agnostic and covers eukaryotes, archaea, and bacteria alike. phyloFlash also provides utilities to visualize multisample comparisons and to integrate the recovered SSU rRNAs in a metagenomics workflow by linking them to MAGs using assembly graph parsing.

Accordingly, the phyloFlash software was obtained from https://github.com/HRGV/phyloFlash and used following the disclosure of (Gruber-Vodicka et al. (2020, phyloFlash: Rapid SSU rRNA profiling and targeted assembly from metagenomes. Novel Syst, Biol. Tech. 5(5):e00920-20) to extract rRNA sequences to get information on the taxonomic composition of the samples. This analysis revealed that the Tromsø samples, particularly Sample 1 (E3-6-3), contained large amounts of an algal specie. Sample 6 (E1-22-3) sample also contains large amounts of a Cilliate. These organisms were not detected in the studies disclosed in Example as those 16S rRNA amplicon primers targeted prokaryotes only. A comparison of rRNA taxonomy across all of the Samples 1-10 (from both the North Atlantic and North Pacific) revealed that many of the most abundant organisms in all of the samples were classified to the same taxonomic orders, for example from Oceanospiralles, Rhodobacteriales and Alteromondales.

### Metagenome Analyses

Metagenomic data was assembled using the metaSPAdes assembler (Nurk et al., 2017, metaSPAdes: a new versatile metagenomic assembler. Genome Res 27:824-834) with the Anvi'o snake-make assembly pipeline (Eren et al., 2015, Anvi'o: an advanced analysis and visualization platform for 'omics data. PeerJ 3:e1319). Genome binning was performed using metabat2 software (Kang et al., 2019, MetaBAT 2: an adaptive binning algorithm for robust and efficient genome reconstruction from metagenome assemblies. PeerJ 7:e7359) and MaxBin2 software (Wu et al., 2014, MaxBin: an automated binning method to recover individual genomes from metagenomes using an expectation-maximization algorithm. Microbiome 2:26). The genome bins were de-replicated using dRep software (Olm et al., 2017, dRep: A tool for fast and accurate genomede-replication that enables tracking of microbial genotypes and improved genome recovery from metagenomes. The ISME J 11:2864-2868), quality of the bins was assessed using the CheckM software (Parks et al., 2015, CheckM: assessing the quality of microbial genomes recovered from isolates, single cells, and metagenomes. Genome Res 25: 1043-1055) and taxonomy was assigned using GTDB-Tk software (Chaumeil et al., 2019, GTDB-Tk: a toolkit to classify genomes with the Genome Taxonomy Database. Bioinformatics 5; 36(6):1925-1927). phyloFlash software (Gruber-Vodicka et al., 2019, phyloFlash—Rapid SSU rRNA profiling and targeted assembly from metagenomes: Supplementary Information. ASM J mSystems 5(5):doi.org/10.1128/mSystems.00920-20) was used to extract rRNA sequences from the metagenome assemblies to generate information on the taxonomic composition of the samples.

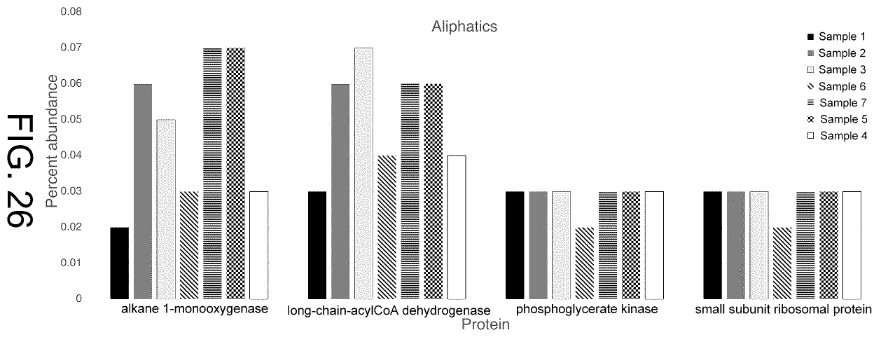

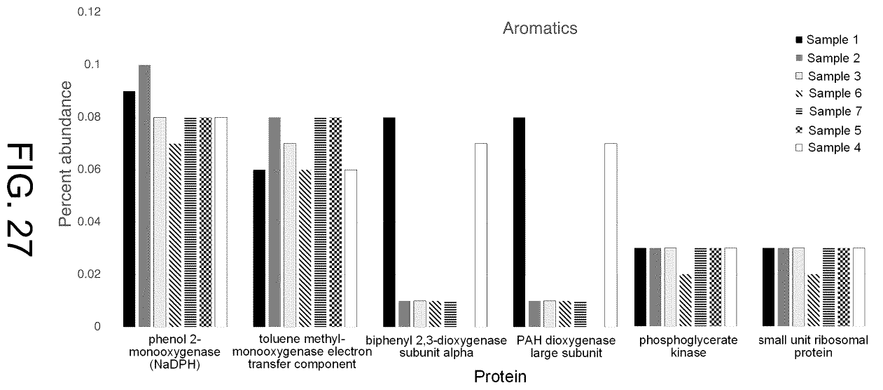

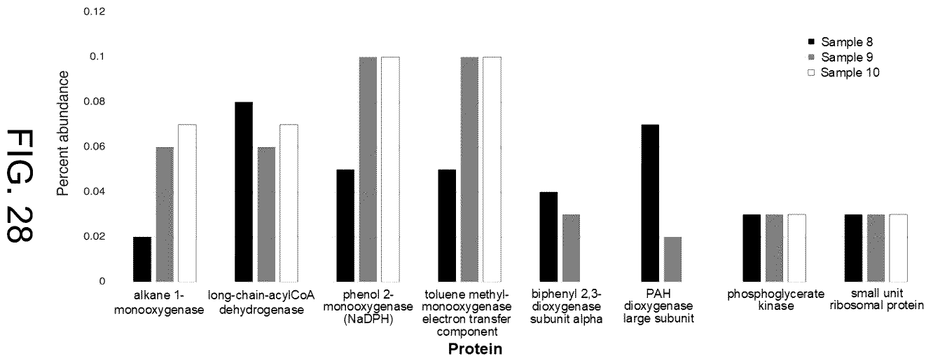

The metagenome assemblies were annotated using the Metaerg annotation pipeline following the teaching of Dong and Strous (2019, An integrated pipeline for annotation and yisualization of metagenomic contigs. Front. Genet. 10:999) with a focus on genes that were involved in hydrocarbon degradation and biofilm production. A selection of annotated genes from the Tromsø North Atlantic samples is shown in FIGS. 26, 27, and a selection of annotated genes from the British Columbia North Pacific samples is shown in FIG. 28.

The relative abundance of two annotated genes involved in degradation of aliphatics, alkane 1-monooxygenase [EC:1.14.153] and long-chain-acyl-CoA dehydrogenase [EC:1.3.8.8], increased over time in Samples 1, 2, 3, and 6 in North Atlantic seawater (FIG. 26). However the relative abundance of these genes remained relatively constant in Samples 4, 5, and 7 as did the two reference housekeeping genes phophoglycerate kinase [EC:2.7.2.3] and small subunit ribosomal protein S7 (FIG. 26).

The relative abundance of two annotated genes involved in degradation of aromatics, phenol 2-monooxygenase (NADPH) [EC:1.14.13.7], and toluene methyl-monooxygenase electron transfer component [EC:1.18.1.3] increased and then decreased in the time-series Samples 1, 2, 3 (FIG. 27). However, these genes and aromatics degrading genes biphenyl 2,3-dioxygenase subunit alpha [EC:1.14.12.18], and PAH dioxygenase large subunit remained constant during the time series Samples 1, 2, and 3 (FIG. 27). Also remaining constant were the two reference housekeeping genes (vii) phophoglycerate kinase [EC:2.7.2.3] and (viii) small subunit ribosomal protein S7.

FIG. 26 shows that Sample 4 from the negative control treatment (Treatment 2 Example 4) that did not receive any ABCM, was most similar to the Sample 1 (treatment 5 Example 4) thereby supporting the observations made with 16S rRNA data reviewed in Example 4, that addition of ABCM enrichments facilitates the rapid development and increasing presence of hydrocarbon-degrading microorganisms in the microbiomes. A similar pattern with increased abundance of genes involved in biofilm formation over time was also apparent, with total % gene abundance increasing from 0.15 to 0.26. Notably, hydrocarbon genes show higher abundance than the housekeeping genes in all the samples, implying that the most abundant organisms have more than one copy of hydrocarbon degradation genes.

The same six annotated genes involved in degradation of aliphatics and aromatics and the two reference housekeeping genes were also annotated for the three British Columbia North Pacific Samples 8, 9, and 10 (FIG. 28). Sample 8 showed a lower abundance of the aliphatic degradation marker gene alkane 1-monooxygenase and higher abundance of PAH dioxygenase, reflecting the higher amounts of aromatics in the diluted bitumen used in Example 5. The enzyme profiles and abundance in Samples 9 and 10 were very similar, except for the loss of two of the four selected aromatic degradation genes (v) biphenyl 2,3-dioxygenase subunit alpha [EC:1.14.12.18], and (vi) PAH dioxygenase large subunit. The metagenome from the biofilm at the bottom of the Sample 10 treatment had highest abundance of biofilm genes with a total % abundance at 0.31 compared to Sample 9 with 0.24 and Sample 8with 0.27. These values were higher than the observations of the biofilm metagenomes from Samples 1 to 7.

### Binning Metagenomes

Contiguous fragments of DNA sequences (contigs) greater than 1,000 were used to build Anvivo-profile databases containing annotation and read coverage information for each of Samples 1 to 10 including 302 Mb of the Tromsø assembly and 119 Mb of the BC assembly.

Based on conserved single copy genes (SCGs), the full Tromsø North Atlantic metagenome contained sequences from 149 bacteria, 2 archaea, and protists (refer to Tables 6-9). The Metabat 2 metagenome binning software tool was used following the teaching of Kang et al. (2019, MetaBAT 2: an adaptive binning algorithm for robust and efficient genome reconstruction from metagenome assemblies. PeerJ 7:e7359 DOI 10.77717/peerj.7359) to bin contigs into genome assembled genomes (MAGs). For the co-assembly of the Tromsø metagenome, 90 bins were identified using MetaBat 2 and among these, 30 bins were of high quality (completeness−5 ×contamination>70). The most highly abundant MAG in the control treatment Sample 4 that did not receive an ABCM enrichment, was a Cycloclasticus bacterium. Sample 1, representing the first time point sequenced of the enriched treatment 5 (Example 4), also had high abundance of Cycloclasticus pugetii, thereby demonstrating similarity between control Sample 4 and the early stages of hydrocarbon degradation in Sample 1 with the ABCM treatment. Samples 1-3 and 5-7 that all received ABCM microbiomes had very high abundance of Alcanivorax borkumensis.

Genome bins from the single-sample assemblies and the co-assembly were compared and dereplicated using the dRep software following the teach of Olm et al. (2017, dRep: A tool for fast and accurate genome de-replication that enables tracking of microbial genotypes and improved genome recovery from metagenomes. ISME J. 11:2864-2868). This resulted in 35 good-quality genomes of which 32 have high quality with (completeness−(5×contamination))<70%. The best quality MAG was Alcanivorax borkumensis with a quality score of 99.6%, and a total of 13 MAGs had quality score >90%.

Based on conserved single copy genes (SCGs), the BC metagenome contained sequences from 76 bacteria. Metabat2 software tool was used to bin contigs into metagenome assembled genomes (MAGs). For the BC North Pacific co-assembly, 34 bins were identified including 17 that were high quality (completeness−5×contamination>70). 92% to 95% of the reads mapped to the 34 genome bins. The most highly abundant MAG in Samples 9 and 10 was Alcanivorax borkumensis. The most highly abundant MAG in Sample 8 was a Porticoccaceae bacterium from the uncultivated 50-400-T64 lineage.

Genome bins from the single-sample assemblies and the co-assembly were compared and dereplicated using the dRep software resulting in 20 good-quality genomes of which, 19 were high quality with (completeness−(5×contamination)) less than 70%. One MAG had quality score at 69%. The best quality MAG in Samples 8, 9, and 19 was Alcanivorax borkumensis with a quality score of 100%. One other MAG classified as Parvibaculum sp002694985, had a score of 100%. A total of 13 MAGs had quality scores greater than 90%, among them two Cycloclasticus pugetii MAGs.

Comparison of all the bins constructed from Tromsø Samples 1 to 7 and BC Samples 8 to 10 resulted in 53 good-quality MAGs, 49 with quality scores greater than 70% and 24 with quality scores greater than 90%. These comparisons revealed that two closely related bacteria were shared by the BC and Tromsø samples; Alcanivorax borkumensis and Porticoccaceae 50-400-T64. These genomes were closely related, but not identical with average nucleotide identity ANI>99%. Both appear to be important hydrocarbon degraders. Detailed analyses of the MAG are reported below.

Genes annotated as alkane 1-monooxygenase [EC:1.14.15.3] (K00496), i.e. alkB, a key gene in alkane degradation, were extracted using the MetaSanity pipeline following the teaching of Neely et al. (2020, MetaSanity: an integrated microbial genome evaluation and annotation pipeline. Bioinformatics 36(15):4341-4344). The extracted sequences were queried against Genbank using Blastp and the five top scoring matches were retrieved. The sequences from the metagenomes and Genbank were aligned a phylogenetic tree that was closely analysed. This analysis revealed that the BC and Tromsø contain several novel AlkB genes with no close relatives in Genbank, demonstrating that these communities contain hydrocarbon-degrading communities that have not previously been described. The MAGs with ‘novel’ AlkB genes were B223msp_mb_bin.11 and E1223msp_mb_bin.4 (Sulfitobacter), BC_comsp_mb_bin.26 and B223msp_mb_bin.15 (Rhodobacteraceae), UF_bottom 22_10msp_mb_bin.1 (Roseovarius sp. 003260265), B223msp_mb_bin.16 (Flavobacteriaceae), DB12210msp_mb_bin.16 (Immundisolibacteraceae), BC_comsp_mb_bin.13 (Gammaproteobacteria UBA5335).

### Alcanivorax

Alcanivorax borkumensis is a cosmopolitan marine bacterial species that uses oil hydrocarbons as its exclusive source of carbon and energy. These bacteria are reported to be hydrocarbon degraders, are non-motile and do not have flagella genes.

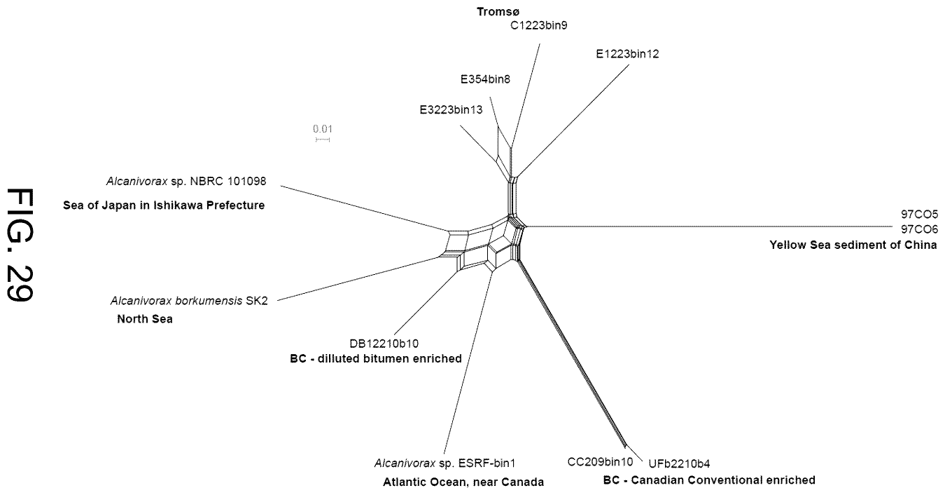

The Alcanivorax genomes constructed from the Tromsø and BC metagenomes were compared to closely related genomes from the North Sea, the Atlantic (near Canada), the Sea of Japan, and the Yellow Sea, using the PanSeq software and following the teaching of Laing et al. (2010, Pan-genome sequence analysis using Panseq: an online tool for the rapid analysis of core and accessory genomic regions. BMC Bioinformatics 11:461-475). The result was an an alignment of 5652 single nucleotide polymorphic sites (SNPs) visualized as a network in FIG. 29 using the SplitsTree software as taught by Huson (1998, Splits Tree: analyzing and visualizing evolutionary data. Bioinformatics 14:68-73).

This network shows that all the Tromsø MAGs are closely related and cluster together. The Alcanivorax genomes from BC fall into two clusters, with the ones from Samples 9 and 10 being identical as expected. The Alcanivorax in BC Sample 8 as distantly related to the Alcanivorax in BC Samples 9 and 10 as it is from the Alcanivorax in Tromsø Samples 1, 2, 3 and 5, 6, 7. The clustering pattern suggests that the oil source is more important than the geographical origin of the seawater used in Examples 4 and 5. This would be most consistent with the Alcanivorax bacteria origination from the hydrocarbons used in the enrichments steps used in Examples 4 and 5. This suggests that the oil source enriched on will be very important when tailoring enrichments for inoculation.

### Porticoccaceae

Porticoccaceae sp., obligate polycyclic aromatic hydrocarbon-degrading bacteria, were observed at high levels in BC Sample 8. i The best quality MAG was DB12210msp_mb_bin.14. A refined version where the contigs were further assembled with Genenious software, has 12 contigs. Annotation with the Metaerg software revealed it carries a very high number of mono- and di-oxygenase genes, such as monooxygenase [EC:1.14.13.-], cyclohexanone monooxygenase [EC:1.14.13.22] and phenol 2-monooxygenase (NADPH) [EC:1.14.13.7]. The annotation of the DB12210mspmb14 genome using the MetaSanity software, also revealed that this genome encodes a “biofilm PGA synthesis protein”, suggesting this organism is also involved in biofilm production. Biofilms observed in the BC Sample 8 treatment.

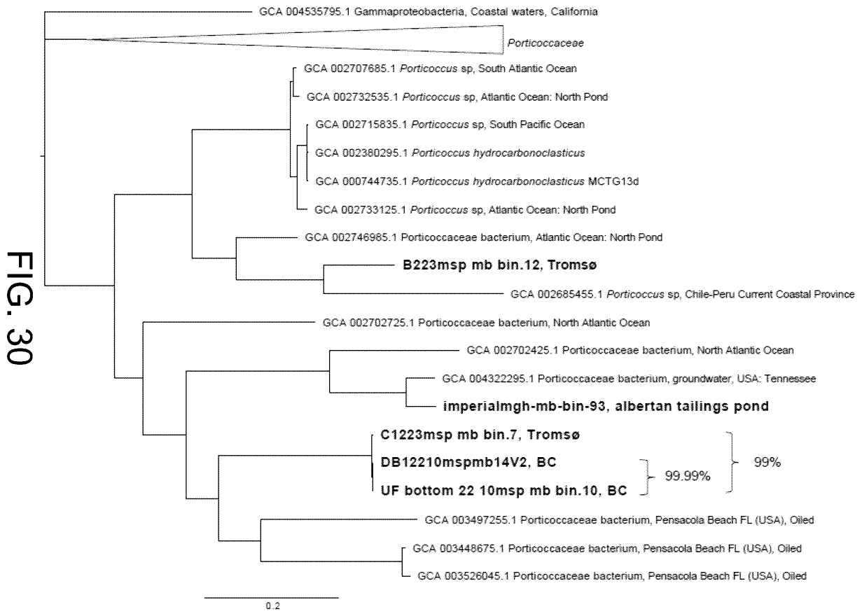

A phylogenetic tree based on conserved single-copy genes using the GtoTree pipeline of the MAGs recovered as disclosed in Examples 4, 5, and 6, and of genomes available in Genbank is shown in FIG. 30. It is noted that the genome from the BC Sample 9 is very similar to the genomes from the Tromsø Samples 1, 2, 3, 5, 6, and 7. Average nucleotide identity was calculated to be 99.03% between the MAG from BC and Tromsø. Thus, this suggests that also this ‘shared’ microorganism might originate from the oil.

### Cycloclasticus

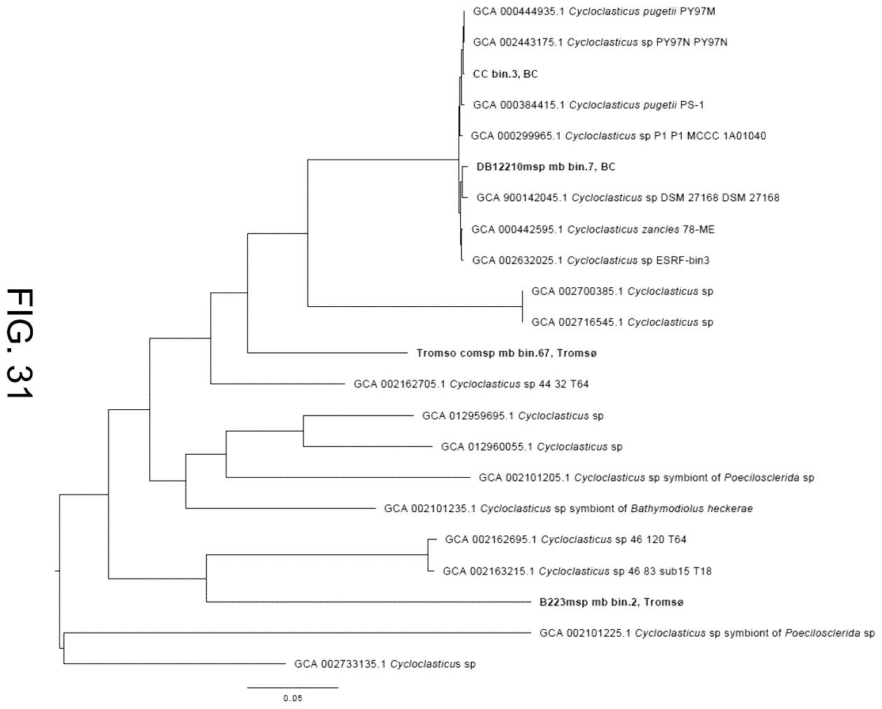

MAGs classified as Cycloclasticus were abundant in both the Tromsø and BC metagenomes. The phylogenetic analyses of conserved proteins from the Cycloclasticus MAGs is shown in FIG. 31. These MAGs were not shared between geographic locations. Distinct MAGs were observed in the Tromsø barrel receiving only oil (B223msp_mb_bin.2) and the barrels with oil and enrichment, suggesting one Cycloclasticus was supplied by the enrichment, while the one in the B-barrel was ‘enriched’ in that barrel from the sea water. It is therefore likely they were enriched from the sea water, not by the added oil.

1. A composition for deployment into marine oil spills, the composition comprising:
a plurality of enriched hydrocarbon-degrading microbiome aggregates; and
a fluid medium wherein the plurality of enriched hydrocarbon-degrading microbiome aggregates is suspended.

2. A composition for deployment into marine oil spills, the composition comprising:
a plurality of enriched hydrocarbon-degrading microbiome aggregates;
a carrier for culturing thereon said plurality of enriched hydrocarbon-degrading microbiome aggregates; and
a fluid medium wherein the selected carrier and the plurality of enriched hydrocarbon-degrading microbiome aggregates are suspended.

3. A composition for deployment into marine oil spills, the composition comprising:
a plurality of enriched hydrocarbon-degrading microbiome aggregates;
a carrier for culturing thereon said plurality of enriched hydrocarbon-degrading microbiome aggregates; and
a fluid medium wherein the selected carrier and the plurality of enriched hydrocarbon-degrading microbiome aggregates are suspended;
whereby the composition is a dynamic microbiome composition wherein numbers of a plurality of microbial species are increasing and/or decreasing.

4. A composition according to any one of claims 1 to 3, additionally comprising one or more selected C₅₋₄₀ hydrocarbons.

5. A composition according to any one of claims 1 to 4, additionally comprising one or more of a surfactant, an emulsifier, an enzyme, a dispersant, and combinations thereof.

6. A composition according to any one of claims 1 to 5, wherein the plurality of enriched hydrocarbon-degrading microbiome aggregates is produced by steps comprising:
collecting a seawater sample from a selected marine water column;
culturing the seawater sample in a liquid medium supplemented one or more selected C₅₋₄₀ hydrocarbons to thereby select and enrich a hydrocarbon-degrading microbiome; and
mixing the selected hydrocarbon-degrading microbiome in the supplemented liquid medium for at least 7 days at a RPM (revolutions per minute) selected from a range of about 0.5 RPM to about 30 RPM to thereby form the plurality of enriched hydrocarbon-degrading microbiome aggregates wherein each of the plurality of enriched hydrocarbon-degrading microbiome aggregates comprises microbiome-produced biofilms extending therethrough and thereabout.

7. A composition according to claim 6, wherein the seawater sample is cultured at a temperature selected from one of about 1° C. to about 5° C. and therebetween, about 4° C. to about 10° C. and therebetween, about 8° C. to about 15° C. and therebetween, about 13° C. to about 20° C. and therebetween, and about 18° C. to about 25° C. and therebetween.

8. A composition according to claim 6 or 7, wherein the marine water column is selected from a hydrocarbon-polluted marine harbour.

9. A composition according to any one of claims 6 to 8, wherein the C₅₋₄₀ hydrocarbon is selected from one or more of a crude oil, a marine fuel, a bunker fuel, a diesel fuel, kerosene, a xylene, a benzene, a toluene, naphthalene, and combinations thereof.

10. A composition according to any one of claims 6 to 9, wherein the liquid medium is selected from one of Buschnell Haas nutrient broth, Väätänen Nine-Salt Solution (VNSS), marine broth, Luria-Bertani marine broth, 2% NaCl Mueller-Hinton broth, and Zobell marine broth.

11. A composition according to any one of claims 6 to 10, wherein the plurality of enriched hydrocarbon-degrading microbiome aggregates comprises one or more of Acanthopleuribacter spp., Acinetobactor spp., Alcanivorax spp., Alteromonadaceae spp., Cycloclasticus spp., Flavobacterium spp., Hyphomonas spp., Marinobacter spp., Porticoccaeae spp., Pseudomonas spp., Rhodobacteraceae spp., Sphingopyxis spp., Sphingobium spp., Xanthobacter spp.

12. A composition according to any one of claims 1 to 11, wherein the fluid medium is a volume of a marine water.

13. A composition according to any one of claims 2 to 12, wherein the carrier is selected from crustacean carcasses, crustacean eggs, crustacean larvae, crustacean juveniles, crustacean fecal matter, crustacean shells, particulate exoskeletons of marine molluscs, particulate shells of marine molluscs, particulate microalgae, particulate macroalgae, clays, zeolites, and combinations thereof.

14. A composition according to any one of claims 2 to 13, wherein the carrier comprises copepod carcasses, copepod eggs, copepod larvae, copepod juveniles, copepod fecal matter, and combinations thereof.

15. A composition according to any one of claims 2 to 14, wherein the carrier comprises krill carcasses, krill eggs, krill larvae, krill juveniles, krill fecal matter, and combinations thereof.

16. A method for producing a composition for deployment into marine oil spills, comprising:
collecting a seawater sample from a selected marine water column;
culturing the seawater sample in a first liquid medium supplemented with one or more C5-40 hydrocarbons to thereby select and enrich a hydrocarbon-degrading microbiome therein;
mixing the selected hydrocarbon-degrading microbiome in the first liquid medium for at least 7 days at a RPM (revolutions per minute) selected from a range of about 0.5 RPM to about 30 RPM to thereby form the plurality of enriched hydrocarbon-degrading microbiome aggregates wherein each of the plurality of enriched hydrocarbon-degrading microbiome aggregates comprises microbiome-produced biofilms extending therethrough and thereabout;
transferring a portion of the plurality of enriched hydrocarbon-degrading microbiome aggregates into a fresh volume of the first liquid medium for at least 7 days at a RPM selected from a range of about 0.5 RPM to about 30 RPM to thereby increase the plurality of enriched hydrocarbon-degrading microbiome aggregates wherein each of the plurality of enriched hydrocarbon-degrading microbiome aggregates comprises microbiome-produced biofilms extending therethrough and thereabout;
optionally repeating the transferring of the portion of the plurality of enriched hydrocarbon-degrading microbiome aggregates into a fresh volume of the first liquid medium, one or more times; and
transferring the plurality of enriched hydrocarbon-degrading microbiome aggregates into a fluid medium wherein the plurality of enriched hydrocarbon-degrading microbiome aggregates is suspended.

17. A method for producing a composition for deployment into marine oil spills, comprising:
collecting a seawater sample from a selected marine water column;
culturing the seawater sample in a first liquid medium supplemented with one or more C₅₋₄₀ hydrocarbons to thereby select and enrich a hydrocarbon-degrading microbiome therein;
mixing the selected hydrocarbon-degrading microbiome in the first liquid medium for at least 7 days at a RPM (revolutions per minute) selected from a range of about 0.5 RPM to about 30 RPM to thereby form the plurality of enriched hydrocarbon-degrading microbiome aggregates wherein each of the plurality of enriched hydrocarbon-degrading microbiome aggregates comprises microbiome-produced biofilms extending therethrough and thereabout;
transferring a portion of the plurality of enriched hydrocarbon-degrading microbiome aggregates into a second liquid medium containing therein a selected carrier; and
culturing the transferred portion of enriched hydrocarbon-degrading microbiome aggregates by mixing at a RPM selected from a range of about 0.5 RPM to about 30 RPM for at least 24 h, to thereby form the composition.

18. A method according to claim 16 or 17, wherein the first liquid medium is selected from Buschnell Haas nutrient broth, Väätänen Nine-Salt Solution (VNSS), marine broth, Luria-Bertani marine broth, 2% NaCl Mueller-Hinton broth, and Zobell marine broth.

19. A method according to any one of claim 16 or 18, wherein the one or more C₅₋₄₀ hydrocarbons is/are selected from crude oils, marine fuels, bunker fuels, diesel fuels, kerosenes, xylenes, benzenes, toluenes, and naphthalene.

20. A method according to any one of claims 16 to 19, wherein the sea water sample is cultured in the first liquid medium at a temperature from a range of about 1° C. to about 10° C.

21. A method according to any one of claims 16 to 19, wherein the sea water sample is cultured in the first liquid medium at a temperature from a range of about 10° C. to about 20° C.

22. A method according to any one of claims 16 to 19, wherein the sea water sample is cultured in the first liquid medium at a temperature from a range of about 15° C. to about 25° C.

23. A method according to any one of claims 16 to 19, wherein the second liquid medium is a marine water sample additionally comprising a C₅₋₄₀ hydrocarbon selected from crude oils, marine fuels, bunker fuels, diesel fuels, kerosenes, xylenes, benzenes, toluenes, and naphthalene.

24. A method according to any one of claims 16 to 20, wherein the second liquid medium additionally comprises one of Buschnell Haas nutrient broth, Väätänen Nine-Salt Solution (VNSS), marine broth, Luria-Bertani marine broth, 2% NaCl Mueller-Hinton broth, and Zobell marine broth.

25. A method according to any one of claims 16 to 24, wherein the plurality of enriched hydrocarbon-degrading microbiome aggregates comprises one or more of Acanthopleuribacter spp., Acinetobactor spp., Alcanivorax spp., Alteromonadaceae spp., Cycloclasticus spp., Flavobacterium spp., Hyphomonas spp., Marinobacter spp., Porticoccaeae spp., Pseudomonas spp., Rhodobacteraceae spp., Sphingopyxis spp., Sphingobium spp., Xanthobacter spp.

26. A method according to any one of claims 16 to 25, wherein the carrier is selected from one of crustacean carcasses, crustacean eggs, crustacean larvae, crustacean juveniles, crustacean fecal matter, crustacean shells, particulate exoskeletons of marine molluscs, particulate shells of marine molluscs, particulate microalgae, particulate macroalgae, clays, zeolites, and combinations thereof.

27. A method for producing hydrocarbon-enriched microbiome aggregate components for use in formulating a dynamic microbiome-based compositions for deployment into marine oil spills, comprising:
collecting a seawater sample from a selected marine water column;
culturing a first portion of the seawater sample in a first liquid medium supplemented with a first selected C₅₋₄₀ hydrocarbon to thereby select and enrich a first hydrocarbon-degrading microbiome therein;
mixing the first enriched hydrocarbon-degrading microbiome in the first liquid medium for at least 7 days at a RPM selected from a range of about 0.5 RPM to about 30 RPM to thereby form the plurality of first enriched hydrocarbon-degrading microbiome aggregates wherein each of the plurality of first enriched hydrocarbon-degrading microbiome aggregates comprises first microbiome-produced biofilms extending therethrough and thereabout; and
maintaining the plurality of first enriched hydrocarbon-degrading microbiome aggregates in a viable condition by repeated transfers of the plurality of first enriched hydrocarbon-degrading microbiome aggregates every 7 days into a fresh first selected liquid medium for a continued gentle mixing a RPM selected from a range of about 0.5 RPM to about RPM.

28. A method according to claim 27, wherein the first liquid medium is one of Buschnell Haas nutrient broth, Väätänen Nine-Salt Solution (VNSS), marine broth, Luria-Bertani marine broth, 2% NaCl Mueller-Hinton broth, and Zobell marine broth.

29. A method according to claim 27, wherein the first selected C₅₋₄₀ hydrocarbon is one of a crude oil, a marine fuel, a bunker fuel, a diesel fuel, kerosene, a xylene, a benzene, a toluene, and naphthalene.

30. A method according to claim 27, wherein the first enriched hydrocarbon-degrading microbiome comprises one or more of Acanthopleuribacter spp., Acinetobactor spp., Alcanivorax spp., Alteromonadaceae spp., Cycloclasticus spp., Flavobacterium spp., Hyphomonas spp., Marinobacter spp., Porticoccaeae spp., Pseudomonas spp., Rhodobacteraceae spp., Sphingopyxis spp., Sphingobium spp., Xanthobacter spp.

31. A method according to claim 27, additionally comprising:
culturing a second portion of the seawater sample in a second liquid medium supplemented with a second selected C₅₋₄₀ hydrocarbon for at least 7 days, to thereby select and enrich a second hydrocarbon-degrading microbiome therein;
mixing the second enriched hydrocarbon-degrading microbiome in the second liquid medium for at least 7 days at a RPM selected from a range of about 0.5 RPM to about 30 RPM to thereby form the plurality of second enriched hydrocarbon-degrading microbiome aggregates wherein each of the plurality of second enriched hydrocarbon-degrading microbiome aggregates comprises second microbiome-produced biofilms extending therethrough and thereabout; and
maintaining the plurality of second enriched hydrocarbon-degrading microbiome aggregates in a viable condition by repeated transfers of the plurality of second enriched hydrocarbon-degrading microbiome aggregates every 7 days into a fresh second selected liquid medium for a continued gentle mixing a RPM selected from a range of about 0.5 RPM to about 30 RPM.

32. A method according to claim 31, wherein the second liquid medium is one of Buschnell Haas nutrient broth, Väätänen Nine-Salt Solution (VNSS), marine broth, Luria-Bertani marine broth, 2% NaCl Mueller-Hinton broth, and Zobell marine broth.

33. A method according to claim 31, wherein the second selected C₅₋₄₀ hydrocarbon is one of a crude oil, a marine fuel, a bunker fuel, a diesel fuel, kerosene, a xylene, a benzene, a toluene, and naphthalene.

34. A method according to claim 31, wherein the second enriched hydrocarbon-degrading microbiome comprises one or more of Acanthopleuribacter spp., Acinetobactor spp., Alcanivorax spp., Alteromonadaceae spp., Cycloclasticus spp., Flavobacterium spp., Hyphomonas spp., Marinobacter spp., Porticoccaeae spp., Pseudomonas spp., Rhodobacteraceae spp., Sphingopyxis spp., Sphingobium spp., Xanthobacter spp.

35. A method according to claim 27, additionally comprising:
culturing a third portion of the seawater sample in a third liquid medium supplemented with a third selected C₅₋₄₀ hydrocarbon at a temperature selected from a range of about 1° C. to about 25° C. for at least 7 days, to thereby select and enrich a third hydrocarbon-degrading microbiome therein;
mixing the third enriched hydrocarbon-degrading microbiome in the third liquid medium for at least 7 days at a RPM selected from a range of about 0.5 RPM to about 30 RPM to thereby form the plurality of third enriched hydrocarbon-degrading microbiome aggregates wherein each of the plurality of third enriched hydrocarbon-degrading microbiome aggregates comprises third microbiome-produced biofilms extending therethrough and thereabout; and
maintaining the plurality of third enriched hydrocarbon-degrading microbiome aggregates in a viable condition by repeated transfers of the plurality of third enriched hydrocarbon-degrading microbiome aggregates every 7 days into a fresh third selected liquid medium for a continued gentle mixing a RPM selected from a range of about 0.5 RPM to about 30 RPM.

36. A method according to claim 35, wherein the third liquid medium is one of Buschnell Haas nutrient broth, Väätänen Nine-Salt Solution (VNSS), marine broth, Luria-Bertani marine broth, 2% NaCl Mueller-Hinton broth, and Zobell marine broth.

37. A method according to claim 35, wherein the third selected C₅₋₄₀ hydrocarbon is one of a crude oil, a marine fuel, a bunker fuel, a diesel fuel, kerosene, a xylene, a benzene, a toluene, and naphthalene.

38. A method according to claim 35, wherein the third enriched hydrocarbon-degrading microbiome comprises one or more of Acanthopleuribacter spp., Acinetobactor spp., Alcanivorax spp., Alteromonadaceae spp., Cycloclasticus spp., Flavobacterium spp., Hyphomonas spp., Marinobacter spp., Porticoccaeae spp., Pseudomonas spp., Rhodobacteraceae spp., Sphingopyxis spp., Sphingobium spp., Xanthobacter spp.

39. A method according to claim 27, additionally comprising:
culturing a fourth portion of the seawater sample in a fourth liquid medium supplemented with a fourth selected C₅₋₄₀ hydrocarbon for at least 7 days, to thereby select and enrich a fourth hydrocarbon-degrading microbiome therein;
mixing the fourth enriched hydrocarbon-degrading microbiome in the third liquid medium for at least 7 days at a RPM selected from a range of about 0.5 RPM to about 30 RPM to thereby form the plurality of fourth enriched hydrocarbon-degrading microbiome aggregates wherein each of the plurality of fourth enriched hydrocarbon-degrading microbiome aggregates comprises fourth microbiome-produced biofilms extending therethrough and thereabout; and
maintaining the plurality of fourth enriched hydrocarbon-degrading microbiome aggregates in a viable condition by repeated transfers of the plurality of fourth enriched hydrocarbon-degrading microbiome aggregates every 7 days into a fresh fourth selected liquid medium for a continued gentle mixing a RPM selected from a range of about 0.5 RPM to about 30 RPM.

40. A method according to claim 39, wherein the fourth liquid medium is one of Buschnell Haas nutrient broth, Väätänen Nine-Salt Solution (VNSS), marine broth, Luria-Bertani marine broth, 2% NaCl Mueller-Hinton broth, and Zobell marine broth.

41. A method according to claim 39, wherein the fourth selected C₅₋₄₀ hydrocarbon is one of a crude oil, a marine fuel, a bunker fuel, a diesel fuel, kerosene, a xylene, a benzene, a toluene, and naphthalene.

42. A method according to claim 39, wherein the fourth enriched hydrocarbon-degrading microbiome comprises one or more of Acanthopleuribacter spp., Acinetobactor spp., Alcanivorax spp., Alteromonadaceae spp., Cycloclasticus spp., Flavobacterium spp., Hyphomonas spp., Marinobacter spp., Porticoccaeae spp., Pseudomonas spp., Rhodobacteraceae spp., Sphingopyxis spp., Sphingobium spp., Xanthobacter spp.

43. A method according to claim 39, wherein the fourth enriched hydrocarbon-degrading microbiome is recultured, mixed again, and maintained again, one or more times.

44. A method of producing a composition for deployment into a marine oil spill, comprising:
combining a plurality of the first enriched hydrocarbon-degrading microbiome aggregates produced according to the method of claim 27 with one or more of a plurality of second enriched hydrocarbon-degrading microbiome aggregates produced according to the method of claim 31, a plurality of third enriched hydrocarbon-degrading microbiome aggregates produced according to the method of claim 35, and a plurality of fourth enriched hydrocarbon-degrading microbiome aggregates produced according to the method of claim 39, transferring the combined pluralities of enriched hydrocarbon-degrading microbiome aggregates into a liquid medium comprising:
a carbon-starved minimal medium;
a carrier selected from crustacean carcasses, crustacean eggs, crustacean larvae, crustacean juveniles, crustacean fecal matter, crustacean shells, particulate exoskeletons of marine molluscs, particulate shells of marine molluscs, particulate microalgae, particulate macroalgae, clays, zeolites, and combinations thereof;
a C₅₋₄₀ hydrocarbon selected from one of a crude oil, a marine fuel, a bunker fuel, a diesel fuel, kerosene, a xylene, a benzene, a toluene, and naphthalene;
mixing the combined pluralities of the enriched hydrocarbon-degrading microbiome aggregates in a RPM selected from a range of about 0.5 RPM to about 30 RPM for at least 24 h to thereby produce the tailored dynamic microbiome-based composition.

45. A method according to claim 44, wherein the liquid medium additionally comprises a selected C₅₋₄₀ hydrocarbon.

46. A method according to claim 44, wherein the carbon-starved minimal medium is one of Buschnell Haas nutrient broth, Väätänen Nine-Salt Solution (VNSS), marine broth, Luria-Bertani marine broth, 2% NaCl Mueller-Hinton broth, and Zobell marine broth.

47. A method according to claim 44, wherein one or more of a surfactant, an emulsifier, an enzyme, a dispersant, and combinations thereof, is added into the composition.

48. A method according to claim 44, additionally comprising the one or more recultured, mixed again, and maintained again fourth enriched hydrocarbon-degrading microbiome.

49. A method according to claim 44, wherein the mixing is performed at a temperature selected from one of about 1° C. to about 10° C., about 10° C. to about 20° C., and about 15° C. to about 25° C.

50. Use of the dynamic microbiome-based composition according to any one of claims 1 to 15, to rapidly degrade and ameliorate a marine oil spill.
